(19) United States
(12) Patent Application Publication
(10) Pub. No.: US 2026/0360531 A1
(43) Pub. Date: Nov. 12, 2026

(54) 3D MEDICAL IMAGING SIMULATION USING GENERATIVE AI

(71) Applicant: NVIDIA CORPORATION, Santa Clara, CA (US)

(72) Inventors: Pengfei Guo, Fairfax, VA (US); Can Zhao, Redmond, WA (US); Dong Yang, Pocatello, ID (US); Yufan He, Philadelphia, PA (US); Vishwesh Nath, Nolensville, TN (US); Ziyue Xu, Reston, VA (US); Daguang Xu, Potomac, MD (US)

(21) Appl. No.: 19/200,561

(22) Filed: May 6, 2025

(51) Int. Cl. G16H 40/67; G16H 15/00; G16H 30/40; G16H 50/50

(52) U.S. Cl. CPC G16H 40/67; G16H 15/00; G16H 30/40; G16H 50/50

(57) ABSTRACT

In various examples, 3D medical images may be generated from a free-form text prompt using a text-conditional diffusion model to extract a latent representation of a 3D volume described in the text prompt, and using a visual decoder of a 3D compression network to decode the latent representation of the 3D volume into a corresponding 3D medical image. To facilitate the use of free-form text prompts, a dataset with 3D medical images and corresponding radiology reports with fixed-format information may be transformed into a diverse set of training data using one or more language models, and the resulting augmented radiology reports may be sampled and used to train the text-conditional diffusion model. As such, the text-conditional diffusion model may be used to generate simulated or synthetic 3D medical images to create or enhance existing datasets.

### BACKGROUND

Text-to-image generation has become a transformative tool in natural image processing, enabling the synthesis of detailed images from descriptive language. This technology has unlocked new possibilities in areas such as creative media, virtual environments, and accessibility tools by allowing users to visualize concepts directly from text inputs. The ability to generate accurate and contextually rich images from text has not only enhanced user interaction but also pushed the boundaries of artificial intelligence in understanding and replicating complex visual scenes. As the technology progresses, it faces the formidable challenge of extending beyond the generation of two-dimensional (2D) natural scenes to the more complex domain of three-dimensional (3D) medical imaging. The synthesis of 3D medical images from textual descriptions presents a unique set of challenges, including higher demands for accuracy, anatomical precision, and the need for medically relevant imagery that adheres to clinical standards. Addressing these challenges not only expands the scope of text-to-image technology but also holds the potential to revolutionize medical diagnostics and personalized treatment planning through tailored, descriptive visualizations of patient-specific conditions.

Building on the role of medical imaging analysis in modern healthcare, the advancement of Deep Learning (DL) models and other machine learning models has greatly enhanced diagnostic and therapeutic practices. These technologies facilitate insights into patient diagnosis, treatment planning, and disease monitoring. Despite these advancements, existing DL techniques for medical imaging continue to encounter formidable challenges. One of the primary challenges is data scarcity. Certain medical conditions, such as rare cancers or less common diseases, are inherently less represented in available datasets. This scarcity hampers the development of models that can accurately generalize to the full spectrum of possible conditions encountered in clinical settings. Another challenge is how to ensure and maintain that the data being processed, which can include medical images and other sensitive patient data, is collected and processed while adhering to ethical standards and complying with data privacy laws and regulations. Furthermore, high human-annotation cost adds another layer of complexity. Unlike annotations for everyday objects in natural images, medical image annotations often require a deeper level of expertise. Subtle features that may be important to accurate diagnosis or treatment planning may need to be meticulously identified and marked by trained professionals, which significantly drives up the time and cost of preparing datasets.

These challenges collectively form significant barriers to developing DL models in the medical field. To address these challenges, the generation of synthetic or simulated medical data has emerged as a promising solution. Synthetic data offers a way to augment limited datasets, reduce reliance on real patient data, and mitigate the high costs associated with manual annotation. By creating artificial yet realistic medical images, synthetic data not only enriches existing datasets but also provides a scalable and ethical alternative for training deep learning models.

Leveraging recent advancements in generative models, the emerging field of generating a 3D volume from textual descriptions introduces an exciting frontier. By translating abstract textual data into detailed medical images, several approaches have demonstrated the ability of generative models to create realistic and clinically relevant volumetric data. This capability not only enhances data diversity but also addresses the aforementioned high costs and privacy issues associated with traditional data collection processes.

Despite notable advancements in text-conditional medical image generation, several important challenges remain insufficiently addressed in existing research. One of the most significant hurdles is the generation of realistic, high-resolution 3D volumes. This task is particularly challenging due to the immense memory demands of existing 3D frameworks, which must handle the substantial data required for high-dimensional representations. To mitigate these memory constraints, some methods adopt hybrid architectures that combine 2D and 3D components. However, relying on 2D networks can lead to continuity problems within the generated 3D medical volumes, disrupting coherence across slices. Existing techniques for 3D volume generation struggle with significant memory limitations—often manifesting as resolution limitations—which hinder realism and clinical applicability. These constraints often compromise precision and consistency, leading to challenges in generating high-quality and reliable medical images. As such, resolution limitations effectively highlight memory constraints that prevent the generation of clinically viable images.

Secondly, existing text-conditional medical image generation models impose rigid constraints on the format of input textual descriptions. For instance, previous approaches limit inputs to predefined structures, such as demographic information combined with a brief clinical impression. While this standardized format simplifies model training, it significantly hinders the model's flexibility and real-world applicability. Since clinical scenarios are inherently diverse, with varying levels of detail, complexity, and focus depending on the patient and the specific diagnostic needs, the rigidity of standardized formats restrict the model's ability to generalize across different contexts and user needs, reducing their effectiveness in handling diverse clinical scenarios and user needs.

As such, there is a need for improved techniques for generating 3D medical images.

### SUMMARY

Embodiments of the present disclosure relate to simulation of 3D medical images using generative AI. Systems and methods are disclosed that synthesize 3D images (e.g., 3D medical images) from textual descriptions using a diffusion model. Unlike previous techniques that rely on fixed-format text input, the present techniques may be used to train the diffusion model to generate 3D medical images from diverse, free-form textual descriptions. By using features derived from free-format (or free-form) text inputs, a much broader distribution in the latent space can be exhibited when compared to those from fixed-format inputs.

In some embodiments, 3D medical images (e.g., representing 3D computed tomography (CT) scans, magnetic resonance imaging (MRI) scans, positron emission tomography (PET) scans) may be generated from a free-form text prompt using a text-conditional diffusion model to extract a latent representation of a 3D volume described in the text prompt, and using a visual decoder of a 3D compression network to decode the latent representation of the 3D volume into a corresponding 3D medical image. The 3D compression network may include the visual encoder that compresses 3D medical images (e.g., 3D CT volumes) into a compact latent space and a visual decoder that decompresses from the latent space back to 3D medical images. The visual encoder may be used to train the text-conditional diffusion model to extract a compact representation of a simulated 3D medical image, and the visual decoder network may be deployed with the trained text-conditional diffusion model to decode simulated 3D medical images.

To facilitate the use of free-form text prompts, a dataset with 3D medical images and corresponding radiology reports with fixed-format information may be transformed into a diverse set of training data using one or more language models. The resulting augmented radiology reports (e.g., comprising generated summaries with original or rephrased demographics, findings, and impressions) may be sampled and paired with the existing 3D medical image data to create various training examples, which may be used to train the text-conditional diffusion model to generate new 3D medical images. By sampling different fields from the augmented radiology reports and varying the resulting training prompts during training, the text-conditional diffusion model should be updated to process inputs that are not confined to a fixed structure.

As such, the text-conditional diffusion model may be used to generate simulated or synthetic 3D medical images from a wide range of textual prompts, and the simulated or synthetic 3D medical images may be used to create or enhance existing datasets and improve the performance of machine learning models trained on those datasets.

### BRIEF DESCRIPTION OF THE DRAWINGS

The present systems and methods for simulation of 3D medical images using generative AI are described in detail below with reference to the attached drawing figures, wherein:

FIG. 1 is a block diagram of an example 3D image generation pipeline, in accordance with some embodiments of the present disclosure;

FIG. 2 is a block diagram of an example training prompt augmentation pipeline, in accordance with some embodiments of the present disclosure;

FIG. 3 is a block diagram of an example training pipeline, in accordance with some embodiments of the present disclosure;

FIG. 4 is a flow diagram illustrating a method of generating simulated 3D medical image data, in accordance with some embodiments of the present disclosure;

FIG. 5A is a block diagram of an example generative language model system suitable for use in implementing at least some embodiments of the present disclosure;

FIG. 5B is a block diagram of an example generative language model that includes a transformer encoder-decoder suitable for use in implementing at least some embodiments of the present disclosure;

FIG. 5C is a block diagram of an example generative language model that includes a decoder-only transformer architecture suitable for use in implementing at least some embodiments of the present disclosure;

FIG. 6 is a block diagram of an example computing device suitable for use in implementing at least some embodiments of the present disclosure; and

FIG. 7 is a block diagram of an example data center suitable for use in implementing at least some embodiments of the present disclosure.

### DETAILED DESCRIPTION

Systems and methods are disclosed relating to simulation of 3D images using generative AI. More specifically, 3D medical images (e.g., representing 3D computed tomography (CT) scans, magnetic resonance imaging (MRI) scans, positron emission tomography (PET) scans) may be generated from a free-form text prompt using a text-conditional diffusion model to extract a latent representation of a 3D volume described in the text prompt, and using a visual decoder of a 3D compression network to decode the latent representation of the 3D volume into a corresponding 3D medical image.

The 3D compression network may include a visual encoder that compresses 3D images (e.g., 3D CT volumes) into a compact latent space and a visual decoder that decompresses from the latent space back to 3D (medical) images. The 3D compression network may be pre-trained (e.g., on an extensive dataset of over 55,000 3D medical volumes) to facilitate robust performance across diverse anatomical structures and imaging conditions. By compressing 3D medical images (e.g., 3D CT volumes) into (and decompressing them from) a compact latent space, this network significantly reduces memory usage while preserving potentially important anatomical details. As such, the visual encoder of the 3D compression network may be used to train the text-conditional diffusion model to extract a compact representation of a simulated 3D image, and the visual decoder of the 3D compression network may be deployed with the trained text-conditional diffusion model to decode simulated 3D images. The compressed latent representations enable more efficient processing and storage, facilitating the generation of high-resolution images without overwhelming computational resources, and improving the ability to handle large data volumes used in, for example and without limitation, high-resolution 3D medical imaging compared to prior techniques.

At a high level, the text-conditional diffusion model may be used to generate realistic 3D images from free-form text descriptions. As a non-limiting example of one or more embodiments of the present disclosure, a dataset with 3D medical images and corresponding radiology reports with fixed-format information may be transformed into a diverse set of training data using one or more language models. For example, an existing 3D medical image may be segmented to identify a list of organs in the 3D medical image, and one or more fields of a corresponding radiology report may be provided to the language model with the list of organs in a prompt that instructs the language model to generate a general description of the 3D medical image (e.g., succinctly summarizing the body regions and anatomical structures) and/or a prompt that instructs the language model to rephrase one or more of the fields of the radiology report (e.g., demographics, findings, impressions). As such, the resulting augmented radiology reports (e.g., comprising generated summaries with original or rephrased demographics, findings, and impressions) may be sampled and paired with the existing 3D medical image to create various training examples, which may be used to train the text-conditional diffusion model to generate new 3D medical images. By sampling different fields from the augmented radiology reports and varying the resulting training prompts during training, the text-conditional diffusion model learns to process inputs that are not confined to a fixed structure.

As a result, the text-conditional diffusion model may be used to interpret a wide range of textual prompts, capturing the richness and variability of clinical narratives. This flexibility allows healthcare professionals to input more personalized and context-specific information compared to prior techniques, facilitating the simulation of more accurate and clinically relevant 3D medical images. Accordingly, the resulting simulated or synthetic 3D images may be used to create or enhance existing datasets—real or synthetic—and improve the performance of machine learning models trained on them. For example, machine learning models in medical applications often depend heavily on accurate, high-quality training data, and using simulated 3D medical images with artifacts (e.g., discontinuities) generated using existing techniques can substantially degrade model performance and lead to reduced diagnostic accuracy. Using the present techniques, any applicable machine learning model may be trained using more accurate and clinically relevant 3D medical images, leading to improved model performance and diagnostic accuracy.

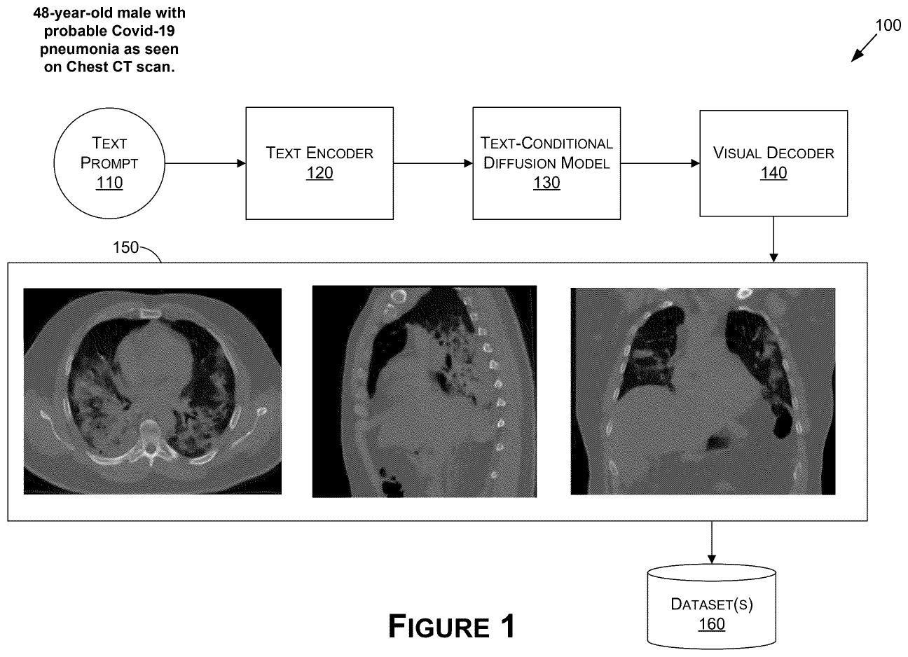

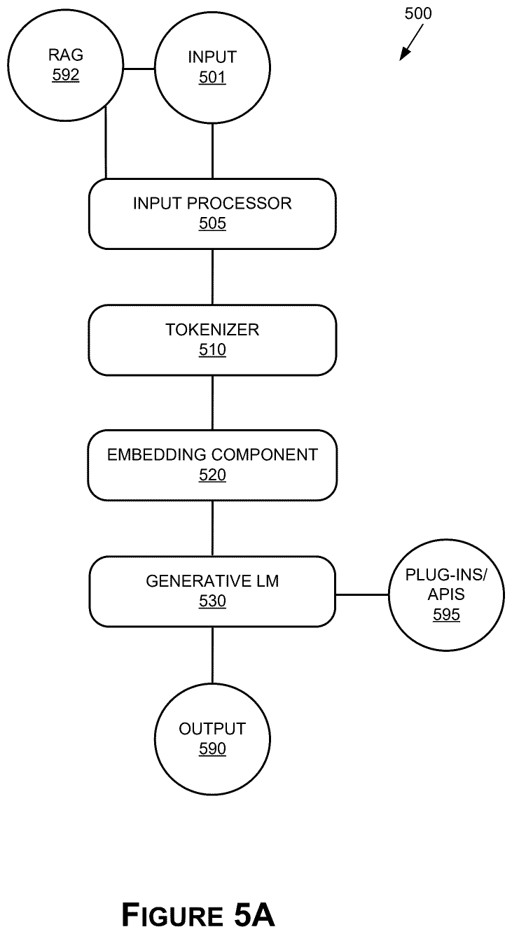

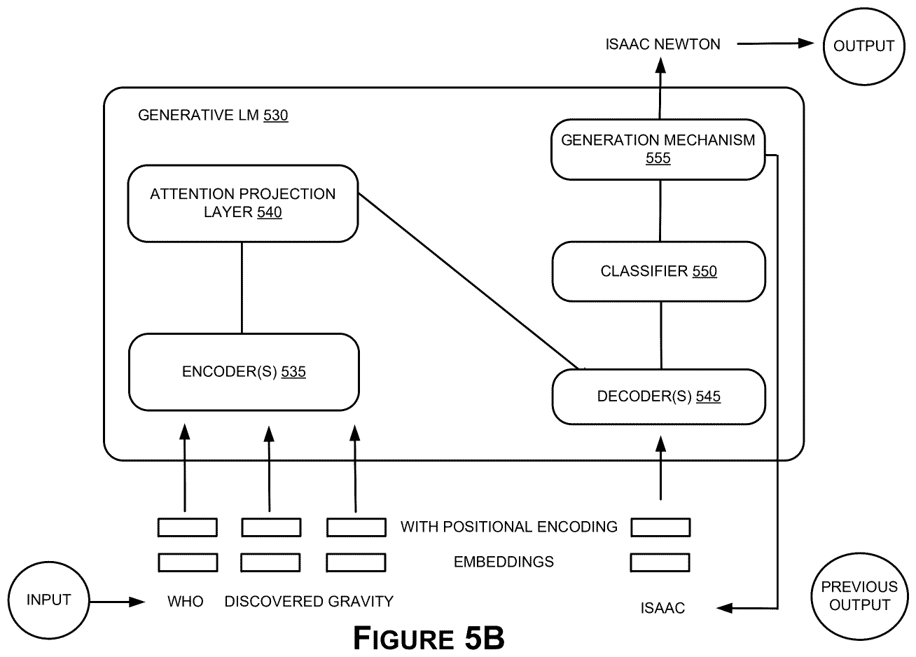

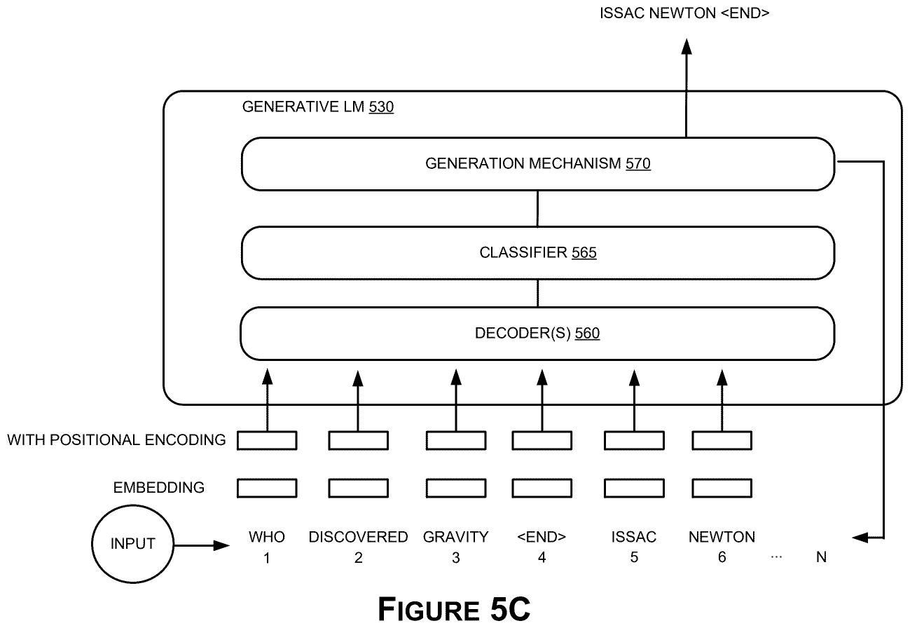

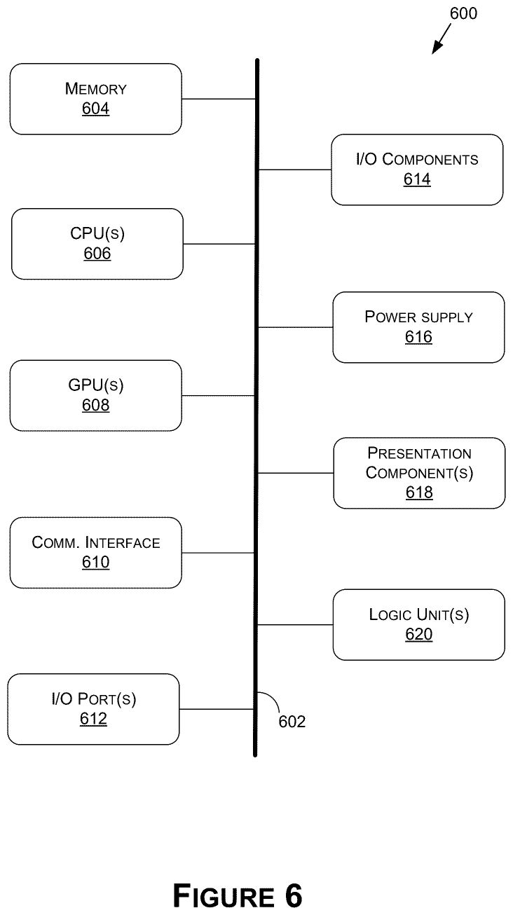

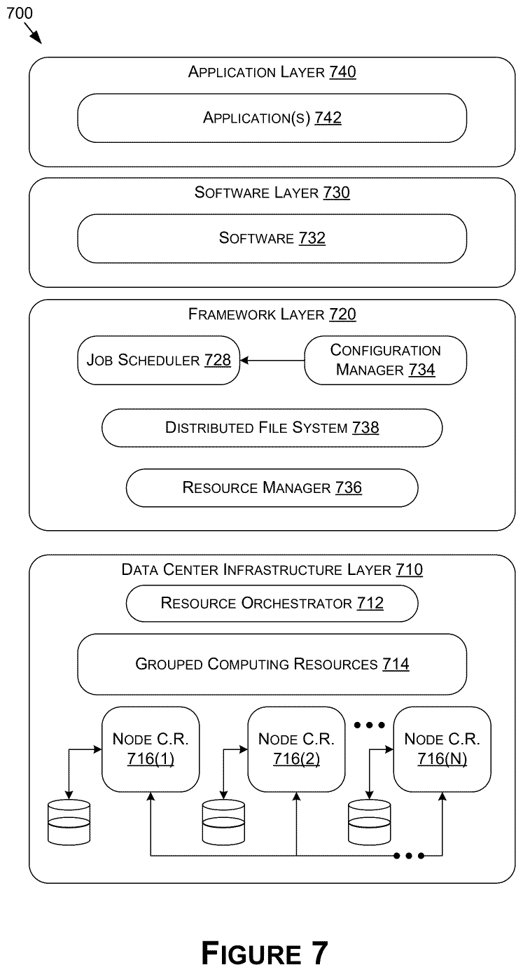

With reference to FIG. 1, FIG. 1 is an example 3D image generation pipeline 100, in accordance with some embodiments of the present disclosure. It should be understood that this and other arrangements described herein are set forth only as examples. Other arrangements and elements (e.g., machines, interfaces, functions, orders, groupings of functions, etc.) may be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by entities may be carried out by hardware, firmware, and/or software. For instance, various functions may be carried out using one or more processors executing instructions stored in one or more memories. For example, in some embodiments, the system and methods described herein may be implemented using one or more generative language models (e.g., as described in FIGS. 5A-5C), one or more computing devices or components thereof (e.g., as described in FIG. 6), and/or one or more data centers or components thereof (e.g., as described in FIG. 7).

At a high level, the 3D image generation pipeline 100 may perform text-conditional 3D volume generation based on a given text prompt T (e.g., the text prompt 110) by learning a mapping function I=F(T) that translates the text prompt T into a realistic 3D (e.g., medical) image I of the descriptive content provided in the text prompt T. Whereas prior techniques often rely on fixed-format text inputs (e.g., {age} years old {sex}:{impression}), which restricts their accessibility, some implementations of the 3D image generation pipeline 100 accept and learn to handle text prompts with free-form text. Additionally or alternatively, unlike prior multi-stage generation approaches that apply super-resolution techniques refining output images to desired dimensions, the 3D image generation pipeline 100 may use a 3D framework to achieve high resolution text-conditional generation. As such, the 3D image generation pipeline 100 may be used to synthesize high-resolution (e.g., 512×512×192 voxels) 3D medical images (e.g., CT volumes) directly from flexible text prompts, significantly enhancing the adaptability of the 3D image generation pipeline 100 to a variety of diverse clinical applications.

More specifically, the 3D image generation pipeline 100 may include a text encoder 120 that generates a text embedding of the text prompt 110, a text-conditional diffusion model 130 that extracts a latent representation of a 3D volume described in the text prompt 110 based on its text embedding, and a visual decoder 140 of a 3D compression network that decodes the latent representation of the 3D volume into a simulated 3D image 150 (e.g., illustrated in FIG. 1 as three orthogonal 2D slices of the 3D volume). Depending on the implementation, the text-conditional diffusion model 130 and/or the visual decoder 140 may be trained to simulate a variety of 3D medical images such as 3D CT, MRI, or PET scans or other suitable 3D volumes. As such, the 3D image generation pipeline 100 may include the simulated 3D image 150 in any applicable dataset (e.g., dataset(s) 160), whether real and/or synthetic.

Generally, the text encoder 120 may use any suitable neural network or machine learning model such as a general-purpose text encoder (e.g., Contrastive Language-Image Pre-Training (CLIP), Text-to-Text Transfer Transformer (T5)) or application-specific (e.g., biomedical-specific) text encoder (e.g., Biomed-CLIP), which may be pre-trained using any known technique, and may be used to encode the text prompt 110 into a text embedding. As such, the text-conditional diffusion model 130 may use the text embedding as conditioning using any known technique (e.g., using cross-attention in one or more layers of the text-conditional diffusion model 130). Although the text encoder 120 is illustrated as a separate component than the text-conditional diffusion model 130, in some scenarios, they may be considered part of the same text-conditional diffusion model.

At a high level, the text-conditional diffusion model 130 may be used to extract a latent representation of realistic 3D medical images conditioned on free-form text input. Diffusion models are a class of probabilistic models designed to approximate a target data distribution p(x) by progressively refining a noise-distorted variable. These models typically function by effectively reversing the dynamics of a predefined Markov Chain across a sequence of T steps. As such, the text-conditional diffusion model 130 may gradually transform random noise into a latent representation of a 3D medical image that aligns with the description given in the text prompt 110 over a series of diffusion steps (time steps). The text-conditional diffusion model 130 may use any suitable architecture such as a time-conditional U-Net, which incorporates a U-Net backbone augmented with time step and text-conditioning embeddings injected at various stages. In this example, the U-Net may act as a series of denoising autoencoders, with each time step in the sequence dedicated to predicting a progressively cleaner version of the latent feature zₜ (a noise-corrupted representation of the original data) over the series of time steps. To adapt to different levels of noise throughout the process, the text-conditional diffusion model 130 may use time embeddings that modify the feature extraction and denoising behavior based on the current time step, allowing the text-conditional diffusion model 130 to adjust its operations as the noise diminishes. For text processing, the text-conditional diffusion model 130 may use cross-attention conditioning mechanisms that align the text embedding with intermediate representations within the time-conditional U-Net, guiding the text-conditional diffusion model 130 toward semantic consistency with the text prompt 110.

A major challenge in generating high-resolution 3D medical images (e.g., CT volumes) is managing the large memory requirements associated with high-dimensional data. As such, in some embodiments, the text-conditional diffusion model 130 extracts latent features in a compact latent space supported by the visual decoder 140 of a 3D compression network, enabling the efficient generation of anatomically precise 3D images while keeping memory requirements relatively low.

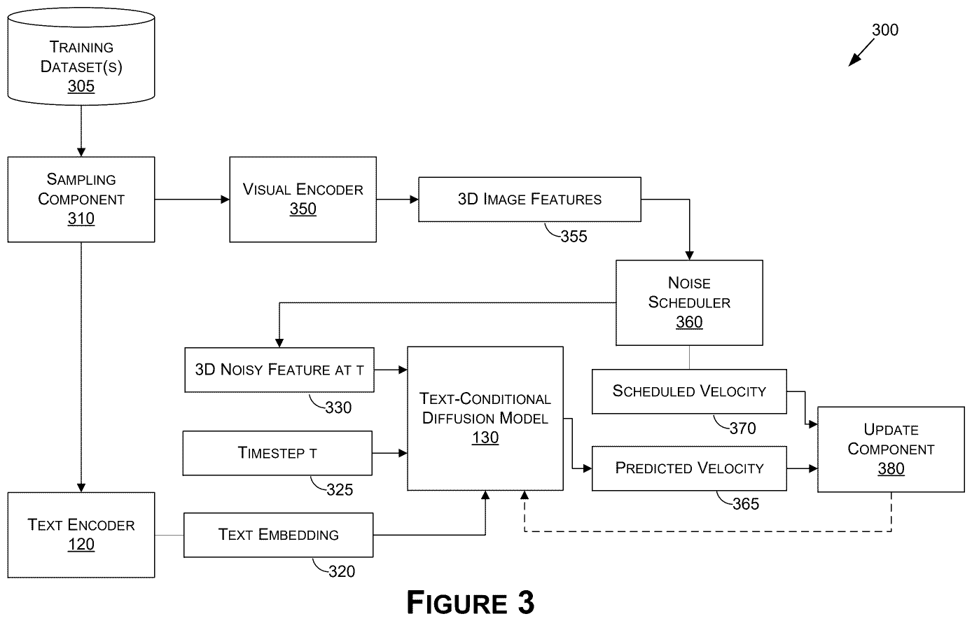

The 3D compression network may include a visual encoder E (e.g., the visual encoder 350 of FIG. 3) and a visual decoder (e.g., the visual decoder 140 of FIG. 1). The visual encoder E may use any known 3D encoder architecture to compress 3D volume data into a low-dimensional latent representation:

$$z = ℰ ( I ) {ϵℝ}^{\frac{H}{4} \times \frac{W}{4} \times \frac{D}{4}} ,$$

and the visual decoder D may use any known 3D decoder architecture to reconstruct the latent representation back to a high-resolution volume:

Î=D(z)=D(ε(I)).

Generating high-resolution 3D volumes presents significant memory challenges within 3D convolutional networks, often easily exceeding graphics processing unit (GPU) memory limitations. Approaches like super-resolution and sliding-window inference help but usually lead to 3D continuity issues or artifacts at boundary transitions. To address this, the visual decoder 140 and/or the visual encoder 350 of the 3D compression network may use tensor splitting parallelism (TSP), which divides large feature maps into smaller segments distributed across multiple GPUs or processed sequentially on a single device, effectively reducing peak memory usage and enhancing inference efficiency. This enables the 3D image generation pipeline 100 to use the visual decoder 140 to generate and process 3D volumes at higher resolutions (e.g., 512×512×192 voxel resolution), facilitating a seamless flow within a unified 3D framework.

Turning now to some potential training techniques, some embodiments train the text-conditional diffusion model 130 of FIG. 1 to accommodate free-form clinical descriptions using augmented radiology reports (e.g., the augmented radiology report 250) generated using the training prompt augmentation pipeline 200.

In a radiology report, the demographics, findings, and impressions sections each serve specific roles in conveying information about the patient and the results of the imaging study. Demographics typically contain patient information such as age, gender, and possibly relevant medical history or symptoms that prompted the imaging study. Findings typically detail what radiologists observed in the images and may include descriptions of normal anatomy and any abnormalities or changes from previous scans. The findings are typically descriptive and may include measurements, locations, and/or characteristics of any lesions or anomalies. Impressions typically summarize the most significant findings and provide an interpretation of what radiologists may indicate in the context of the patient's health. Existing models are typically restricted by rigid text formats with pre-defined text fields, limiting their ability to capture the nuances of diverse clinical scenarios. However, free-form text input is often important for simulating clinically relevant 3D medical images.

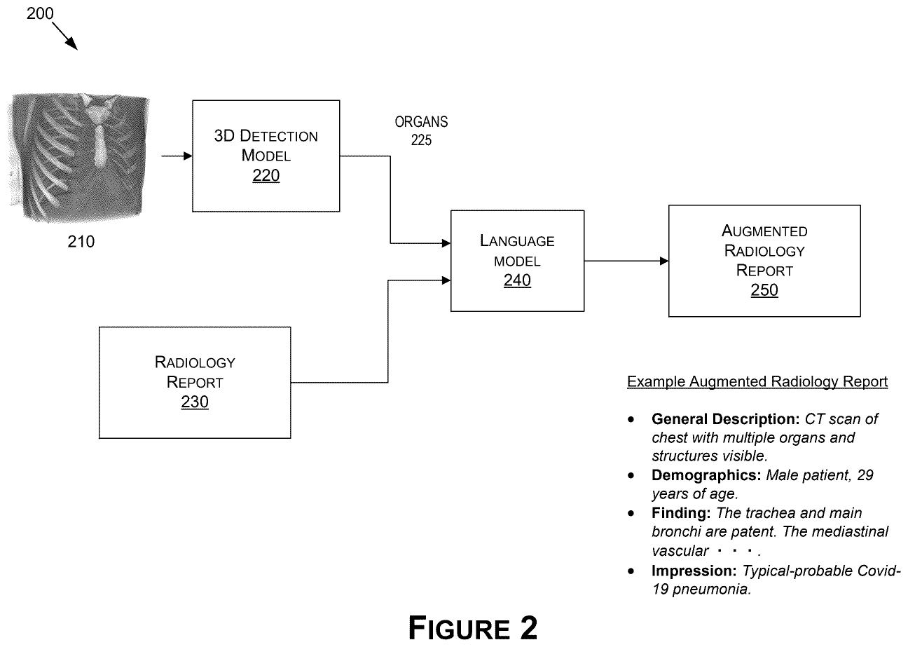

As such, to facilitate the use of free-form text prompts, a dataset(s) comprising 3D medical images and corresponding radiology reports with fixed-format information may be transformed into a diverse set of training data using one or more language models, such as the generative language models described in FIGS. 5A-5C. FIG. 2 is a block diagram of an example training prompt augmentation pipeline 200, in accordance with some embodiments of the present disclosure. At a high level, an existing 3D medical image 210 may be segmented or otherwise processed (e.g., using the 3D detection model 220) to identify a list of organs 225 in the 3D medical image 210, and one or more fields of a corresponding radiology report 230 may be provided to a language model 240 (e.g., with the list of organs 225) in a prompt that instructs the language model 240 to generate a general description of the 3D medical image 210 (e.g., succinctly summarizing the body regions and anatomical structures) and/or a prompt that instructs the language model 240 to rephrase one or more of the fields of the radiology report 230 (e.g., demographics, findings, impressions). As such, the language model 240 may be used to generate a general description and/or augmented variants of demographics, findings, and/or impressions based on existing radiology reports (e.g., the radiology report 230) and/or a list of organs (e.g., organs 225) derived from corresponding segmentation maps, and the language model 240 may be used to transform the radiology report 230 into an augmented radiology report 250 (e.g., comprising generated summaries with original or rephrased demographics, findings, and impressions).

Taking a 3D medical image such as the 3D medical image 210 as an example, the 3D medical image 210 may form a volume Iϵ [symbol] ^{H×W×D} (e.g., in grayscale voxel space), where H denotes the height, W the width, and D the depth, respectively. The 3D medical image 210 may be part of a dataset that pairs the 3D medical image 210 with a corresponding radiology report T (e.g., the radiology report 230). These reports typically include detailed sections on patient demographics T_{d}, findings T_{f}, and impressions Tᵢ, although not all reports include all three sections.

In some embodiments, a 3D detection model 220 may be used to extract a list of organs 225 from the 3D medical image 210. Generally, the 3D detection model 220 may use any known 3D detection, classification, or segmentation technique to identify organs present in the 3D medical image 210. Taking CT scans as an example, the 3D detection model 220 may include a whole-body CT segmentation model such as TotalSegmentator or VISTA3D that accepts a 3D CT volume as input and outputs voxel-level predictions (e.g., assigning a class label to each voxel in 3D space) and/or corresponding segmentation masks for various anatomical structures. As such, models such as these may be used to identify specific organs within the 3D medical image 210, the list of organs may be used to support the creation of a general description T_{g} of the 3D medical image 210 (e.g., succinctly summarizing the body regions and anatomical structures present) and/or a rephrased variation of one or more fields of the radiology report 230. This process facilitates more targeted and contextually relevant text-to-image generation by defining the precise areas covered in the 3D medical image 210.

As such, the prompt augmentation pipeline 200 may form a variety of prompts that instruct the language model 240 to create a general description T_{g} of the 3D medical image 210 and/or rephrase one or more fields of the radiology report 230. Generally, the prompt augmentation pipeline 200 may use any known prompt engineering technique (e.g., using system prompts, role-based prompts, instruction prompts, question prompts, multi-turn prompts, etc.) to provide the language model 240 with an applicable prompt, and the language model 240 may use any known architecture such as those described with respect to FIGS. 5A-5C.

By way of nonlimiting example, an instruction to create a general description of a 3D CT volume based on patient demographics and impressions may be included in a prompt such as:
{“role”: “system”, “content”:
“You are an expert medical assistant AI capable of summarizing clinical documents to user specifications. Please directly give one concise answer without any introduction or explanation, so users can directly save your output as the answer.”},
{“role”: “user”, “content”:
“You have a Chest CT scan containing the following demographics: {demographics} The corresponding impression is as follows: {impression} Act as a user of a 3D volume generation model and write a concise description for this CT scan using one sentence that can be used as the text input. This description should be free-format. You should try to simulate the possible human input. The descriptions must not more than 20 words. Extra words will be ignored.”}

In another non-limiting example, an instruction to rephrase a field sampled from an existing radiology report may be included in a prompt such as:
{role”: “system”, “content”:
“You are an expert medical assistant AI capable of modifying clinical documents to user specifications. You make minimal changes to the original document to satisfy user requests. You never add information that is not already directly stated in the original document. Please directly give one concise answer without any introduction or explanation, so users can directly save your output as the answer.”},
{“role”: “user”, “content”:
“Please rephrase the following sentence: {text}.”}
   These prompts are meant simply as examples, and variations may be implemented within the scope of the present disclosure.

As such, the prompt augmentation pipeline 200 may iteratively sample from a dataset of radiology reports, insert the sampled text (e.g., demographics, findings, and/or impressions) into a corresponding prompt, apply the prompt to the language model 240, and compose a corresponding augmented radiology report 250. In some embodiments, the prompt augmentation pipeline 200 may insert the text generated by the language model 240 into a corresponding field of the augmented radiology report 250. As such, the prompt augmentation pipeline 200 may use the language model 240 to convert structured radiology reports into augmented variants encompassing varied levels of detail and language styles. This textual data creation process may be defined as follows:

T_{g},\hat{T}_{d},\hat{T}_{f},Îᵢ=LLM(O,T_{d},T_{f},Tᵢ),

where T̅_{d}, \hat{T}_{f}, \hat{T}ᵢ and denote the augmented variants of T_{d}, findings T_{f}, and impressions Tᵢ, respectively. Accordingly, the prompt augmentation pipeline 200 may pair each augmented radiology report with its corresponding 3D medical image, repeating the process to produce a training dataset.

As such, the augmented radiology reports may be sampled and used with the corresponding 3D medical images as training pairs to train the text-conditional diffusion model 130. FIG. 3 is a block diagram of an example training pipeline 300 that may be used to train the text-conditional diffusion model 130, in accordance with some embodiments of the present disclosure. In this example, the training pipeline 300 includes the text encoder 120 and the text-conditional diffusion model 130 of FIG. 1 and the visual encoder 350 of the 3D compression network corresponding to the visual decoder 140 of FIG. 1. At a high level, the training pipeline 300 may include a sampling component 310 that samples text from fields of radiology reports (e.g., augmented radiology reports generated using the prompt augmentation pipeline 200 of FIG. 2) from one or more training dataset(s) 305, applies the sampled text to the text encoder 120 to extract a text embedding 320, and applies the corresponding 3D medical image to the visual encoder 350 to extract 3D image features 355 (in any suitable order). In one or more embodiments, the text embedding 320 and 3D image features 355 may be used to train the text-conditional diffusion model 130, as explained in more detail below.

In some embodiments, the visual decoder 140 and/or the visual encoder 350 of the 3D compression network may be pre-trained using a dataset comprising a desired type of 3D image to simulate (e.g., CT scans, MRI scans, PET scans). The 3D images in the dataset may be augmented using any known technique. For example, CT intensities may be clipped (e.g., to a Hounsfield Unit range of −1000 to 1000) and normalized (e.g., to [0,1]), or magnetic resonance (MR) intensities may be scaled (e.g., scaling the 0th to 99.5th percentile range to [0,1]) and enhanced with augmentations such as random bias fields, Gibbs noise, contrast adjustments, and/or histogram shifts. Example spatial augmentations include random flipping, rotation, intensity scaling, shifting, and/or resolution changes. In one or more embodiments, the 3D compression network may be trained to reconstruct the 3D images in the (e.g., augmented) dataset using any known technique. In an example implementation, the 3D compression network may be trained in two phases: first, for some number of training iterations and/or passes through the dataset (e.g., 100 epochs) on (e.g., [64,64,64] voxel) patches to enhance generalization for partial volume effects, and then for some number of training iterations and/or passes through the dataset (e.g., 200 epochs) on (e.g., [128,128,128] voxel) patches to capture richer contextual information, improving overall performance.

In some embodiments, the training process for the text-conditional diffusion model 130 may include a series of data pre-processing for optimized learning. For example, 3D medical images in the training dataset(s) 305 may be aligned (e.g., using the Right-Anterior-Superior (RAS) axis code such that the axes correspond to Right-Left, Anterior-Posterior, and Superior-Inferior directions), normalized (e.g., by scaling their intensity values from −1000 to 1000 into [0,1]), resampled into a designated voxel spacing (e.g., 0.75×0.75×1.5 millimeters), and/or cropped (e.g., to 512×512×192 voxels, using trilinear interpolation). In one or more embodiments, one or more (e.g., pre-processed) 3D medical image(s) may be passed through the (e.g., pre-trained) visual encoder 350 to generate a corresponding compressed latent representation (e.g., the 3D image features 355). In some embodiments, 3D medical images from the training dataset(s) 305 may be encoded during preprocessing, and the resulting 3D image features may be associated with the corresponding augmented radiology reports (or sampled fields thereof) in the training dataset(s) 305.

As such, the sampling component 310 may sample from the fields of the (e.g., augmented) radiology reports and apply corresponding training pairs in an order that introduces variability. For example, the augmented radiology reports may include fields such as general description, demographics, findings, and/or impressions, and the sampling component 310 may randomly sample from the fields and use a corresponding training pair (e.g., a sampled text prompt, 3D medical image, pre-encoded 3D image features) to train the text-conditional diffusion model 130. Taking augmented radiology reports with four fields as an example, the sampling component 310 may randomly select n∈{1, 2, 3, 4} prompt types from the four fields, generate corresponding training pairs, shuffle their ordering to include diverse combinations, and/or randomly replace sections of original text T in a radiology report with corresponding rephrased text (e.g., generated using the prompt augmentation pipeline 200 of FIG. 2). By sampling different fields from the radiology reports and varying the resulting training prompts during training, the text-conditional diffusion model 130 should learn to handle various prompt structures, improving its robustness in generating outputs from diverse textual inputs (e.g., interpreting rich clinical narratives beyond fixed templates) and substantially enhancing both the quality of the generated 3D medical images and their alignment with the corresponding text descriptions compared to prior techniques.

In some embodiments, the text-conditional diffusion model 130 may be trained using velocity prediction loss so the text-conditional diffusion model 130 learns to predict the velocity ν (e.g., the predicted velocity 365) between noisy and clean states, allowing it to handle the denoising sequence more smoothly. This approach improves stability, sample quality, and training efficiency, making it more effective in reconstructing high-fidelity data through the diffusion process.

More specifically, a noise scheduler 360 may use any known technique to systematically add noise to the 3D image features 355 to generate a corresponding 3D noisy feature 330 and compute a corresponding scheduled velocity 170 at step T, and the text-conditional diffusion model 130 may iteratively reduce this noise (e.g., using a loss function such as L2). The noise scheduler 360 may effectively simulate realistic noisy inputs by progressively adding noise to the latent feature z₀ (e.g., the 3D image features 355), gradually increasing noise over a sequence of time (diffusion) steps. For any given time step T, the text-conditional diffusion model 130 may accept as input the 3D noisy feature 330 produced by the noise scheduler 360 for that time step and a representation of the time step 325 (e.g., an embedding that encodes the current time step T), and the text-conditional diffusion model 130 may generate a predicted velocity 365, conditioned on the text embedding 320. The update component 380 may compute the difference between the predicted velocity 365 and the scheduled velocity 170 and use the difference to update the text-conditional diffusion model 130. By way of non-limiting example, the update component 380 may use a loss function such as:

$${L}_{Θ} = {𝔼}_{ℰ ( I ) , t , {c}_{text}} [ {‖ v - {v}_{θ} ( {z}_{t} , t , {c}_{text} ) ‖}_{2}^{2} ] ,$$

where the neural backbone ν_{θ} may be conditioned on time step t and the text conditions as cₜₑₓₜ. Mixed precision training and/or gradient scaling may be used to optimize memory and computational efficiency, improving the ability of the text-conditional diffusion model 130 to handle the complexity of the data and training process effectively.

As such, the training pipeline 300 may be used to train the text-conditional diffusion model 130 to learn a mapping function that translates an input text prompt (e.g., the text prompt 110 of FIG. 1) into a realistic 3D medical image (e.g., the simulated 3D image 150 of FIG. 1). As such, the 3D image generation pipeline 100 of FIG. 1 may be used to generate and include any number of simulated (or synthetic) 3D medical images in the dataset(s) 160, which may include real and/or simulated data.

Using the present techniques, any applicable machine learning model may be trained using more accurate and clinically relevant 3D medical images, leading to improved model performance and diagnostic accuracy. Depending on the application, the (e.g., simulated 3D medical images in) the dataset(s) 160 may be used to train a variety of models and improve their accuracy. Example machine learning models that may benefit significantly from more accurate simulated 3D medical images include 3D segmentation models (e.g., that detect anatomical structures or pathologies), 3D object detection models (e.g., that localize structures such as nodules, lesions, or organs), 3D generative models (e.g., 3D generative adversarial networks or diffusion models that synthesize new images or complete missing data), and/or otherwise.

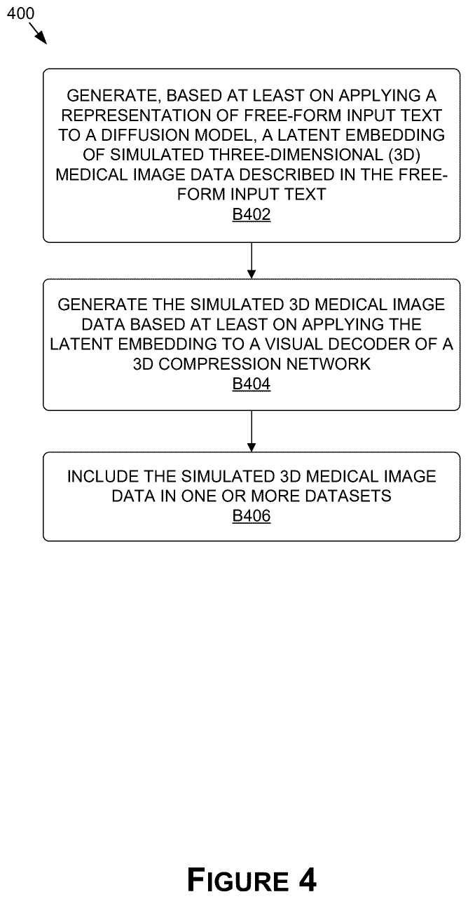

Now referring to FIG. 4, each block of method 400, described herein, comprises a computing process that may be performed using any combination of hardware, firmware, and/or software. For instance, various functions may be carried out using one or more processors executing instructions stored in one or more memories. The method 400 may also be embodied as computer-usable instructions stored on computer storage media. The method 400 may be provided by a standalone application, a service or hosted service (standalone or in combination with another hosted service), as a microservice via an application programming interface (API) or a plug-in to another product, to name a few. In addition, method 400 is described, by way of example, with respect to the 3D image generation pipeline 100 of FIG. 1. However, this method may additionally or alternatively be executed by any one system, or any combination of systems, including, but not limited to, those described herein.

FIG. 4 is a flow diagram showing a method 400 for generating simulated 3D medical image data, in accordance with some embodiments of the present disclosure. The method 400, at block B402, includes generating, based at least on applying a representation of free-form input text to a diffusion model, a latent embedding of simulated three-dimensional (3D) medical image data described in the free-form input text. For example, with respect to the 3D image generation pipeline 100 of FIG. 1, the text encoder 120 may generate a text embedding of the text prompt 110, and the text-conditional diffusion model 130 may extract a latent representation of a 3D volume described in the text prompt 110 based on its text embedding.

The method 400, at block B404, includes generating the simulated 3D medical image data based at least on applying the latent embedding to a visual decoder of a 3D compression network. For example, with respect to the 3D image generation pipeline 100 of FIG. 1, the visual decoder 140 may decode the latent representation of the 3D volume into the simulated 3D image 150 (e.g., illustrated in FIG. 1 as three orthogonal 2D slices of the 3D volume).

The method 400, at block B406, includes storing the simulated 3D medical image data in one or more datasets. For example, with respect to the 3D image generation pipeline 100 of FIG. 1, the 3D image generation pipeline 100 may include the simulated 3D image 150 in any applicable dataset (e.g., dataset(s) 160), whether real and/or synthetic.

### Example Language Models

In at least some embodiments, language models, such as large language models (LLMs), vision language models (VLMs), multi-modal language models (MMLMs), and/or other types of generative artificial intelligence (AI) may be implemented. These models may be capable of understanding, summarizing, translating, and/or otherwise generating text (e.g., natural language text, code, etc.), images, video, computer aided design (CAD) assets, OMNIVERSE and/or METAVERSE file information (e.g., in USD format, such as OpenUSD), and/or the like, based on the context provided in input prompts or queries. These language models may be considered “large,” in embodiments, based on the models being trained on massive datasets and having architectures with large number of learnable network parameters (weights and biases)—such as millions or billions of parameters. The LLMs/VLMs/MMLMs/etc. may be implemented for summarizing textual data, analyzing and extracting insights from data (e.g., textual, image, video, etc.), and generating new text/image/video/etc. in user-specified styles, tones, and/or formats. The LLMs/VLMs/MMLMs/etc. of the present disclosure may be used exclusively for text processing, in embodiments, whereas in other embodiments, multi-modal LLMs may be implemented to accept, understand, and/or generate text and/or other types of content like images, audio, 2D and/or 3D data (e.g., in USD formats), and/or video. For example, vision language models (VLMs), or more generally multi-modal language models (MMLMs), may be implemented to accept image, video, audio, textual, 3D design (e.g., CAD), and/or other inputs data types and/or to generate or output image, video, audio, textual, 3D design, and/or other output data types.

Various types of LLMs/VLMs/MMLMs/etc. architectures may be implemented in various embodiments. For example, different architectures may be implemented that use different techniques for understanding and generating outputs—such as text, audio, video, image, 2D and/or 3D design or asset data, etc. In some embodiments, LLMs/VLMs/MMLMs/etc. architectures such as recurrent neural networks (RNNs) or long short-term memory networks (LSTMs) may be used, while in other embodiments transformer architectures—such as those that rely on self-attention and/or cross-attention (e.g., between contextual data and textual data) mechanisms—may be used to understand and recognize relationships between words or tokens and/or contextual data (e.g., other text, video, image, design data, USD, etc.). One or more generative processing pipelines that include LLMs/VLMs/MMLMs/etc. may also include one or more diffusion block(s) (e.g., denoisers). The LLMs/VLMs/MMLMs/etc. of the present disclosure may include encoder and/or decoder block(s). For example, discriminative or encoder-only models like BERT (Bidirectional Encoder Representations from Transformers) may be implemented for tasks that involve language comprehension such as classification, sentiment analysis, question answering, and named entity recognition. As another example, generative or decoder-only models like GPT (Generative Pretrained Transformer) may be implemented for tasks that involve language and content generation such as text completion, story generation, and dialogue generation. LLMs/VLMs/MMLMs/etc. that include both encoder and decoder components like T5 (Text-to-Text Transformer) may be implemented to understand and generate content, such as for translation and summarization. These examples are not intended to be limiting, and any architecture type—including but not limited to those described herein—may be implemented depending on the particular embodiment and the task(s) being performed using the LLMs/VLMs/MMLMs/etc.

In various embodiments, the LLMs/VLMs/MMLMs/etc. may be trained using unsupervised learning, in which an LLMs/VLMs/MMLMs/etc. learns patterns from large amounts of unlabeled text/audio/video/image/design/USD/etc. data. Due to the extensive training, in embodiments, the models may not require task-specific or domain-specific training. LLMs/VLMs/MMLMs/etc. that have undergone extensive pre-training on vast amounts of unlabeled data may be referred to as foundation models and may be adept at a variety of tasks like question-answering, summarization, filling in missing information, translation, image/video/design/USD/data generation. Some LLMs/VLMs/MMLMs/etc. may be tailored for a specific use case using techniques like prompt tuning, fine-tuning, retrieval augmented generation (RAG), adding adapters (e.g., customized neural networks, and/or neural network layers, that tune or adjust prompts or tokens to bias the language model toward a particular task or domain), and/or using other fine-tuning or tailoring techniques that optimize the models for use on particular tasks and/or within particular domains.

In some embodiments, the LLMs/VLMs/MMLMs/etc. of the present disclosure may be implemented using various model alignment techniques. For example, in some embodiments, guardrails may be implemented to identify improper or undesired inputs (e.g., prompts) and/or outputs of the models. In doing so, the system may use the guardrails and/or other model alignment techniques to either prevent a particular undesired input from being processed using the LLMs/VLMs/MMLMs/etc., and/or preventing the output or presentation (e.g., display, audio output, etc.) of information generating using the LLMs/VLMs/MMLMs/etc. In some embodiments, one or more additional models—or layers thereof—may be implemented to identify issues with inputs and/or outputs of the models. For example, these “safeguard” models may be trained to identify inputs and/or outputs that are “safe” or otherwise okay or desired and/or that are “unsafe” or are otherwise undesired for the particular application/implementation. As a result, the LLMs/VLMs/MMLMs/etc. of the present disclosure may be less likely to output language/text/audio/video/design data/USD data/etc. that may be offensive, vulgar, improper, unsafe, out of domain, and/or otherwise undesired for the particular application/implementation.

In some embodiments, the LLMs/VLMs/etc. may be configured to or capable of accessing or using one or more plug-ins, application programming interfaces (APIs), databases, data stores, repositories, etc. For example, for certain tasks or operations that the model is not ideally suited for, the model may have instructions (e.g., as a result of training, and/or based on instructions in a given prompt) to access one or more plug-ins (e.g., 3^{rd} party plugins) for help in processing the current input. In such an example, where at least part of a prompt is related to restaurants or weather, the model may access one or more restaurant or weather plug-ins (e.g., via one or more APIs) to retrieve the relevant information. As another example, where at least part of a response requires a mathematical computation, the model may access one or more math plug-ins or APIs for help in solving the problem(s), and may then use the response from the plug-in and/or API in the output from the model. This process may be repeated—e.g., recursively—for any number of iterations and using any number of plug-ins and/or APIs until a response to the input prompt can be generated that addresses each ask/question/request/process/operation/etc. As such, the model(s) may not only rely on its own knowledge from training on a large dataset(s), but also on the expertise or optimized nature of one or more external resources—such as APIs, plug-ins, and/or the like.

In some embodiments, multiple language models (e.g., LLMs/VLMs/MMLMs/etc., multiple instances of the same language model, and/or multiple prompts provided to the same language model or instance of the same language model may be implemented, executed, or accessed (e.g., using one or more plug-ins, user interfaces, APIs, databases, data stores, repositories, etc.) to provide output responsive to the same query, or responsive to separate portions of a query. In at least one embodiment, multiple language models e.g., language models with different architectures, language models trained on different (e.g. updated) corpuses of data may be provided with the same input query and prompt (e.g., set of constraints, conditioners, etc.). In one or more embodiments, the language models may be different versions of the same foundation model. In one or more embodiments, at least one language model may be instantiated as multiple agents—e.g., more than one prompt may be provided to constrain, direct, or otherwise influence a style, a content, or a character, etc., of the output provided. In one or more example, non-limiting embodiments, the same language model may be asked to provide output corresponding to a different role, perspective, character, or having a different base of knowledge, etc.—as defined by a supplied prompt.

In any one of such embodiments, the output of two or more (e.g., each) language models, two or more versions of at least one language model, two or more instanced agents of at least one language model, and/or two more prompts provided to at least one language model may be further processed, e.g., aggregated, compared or filtered against, or used to determine (and provide) a consensus response. In one or more embodiments, the output from one language model—or version, instance, or agent—maybe be provided as input to another language model for further processing and/or validation. In one or more embodiments, a language model may be asked to generate or otherwise obtain an output with respect to an input source material, with the output being associated with the input source material. Such an association may include, for example, the generation of a caption or portion of text that is embedded (e.g., as metadata) with an input source text or image. In one or more embodiments, an output of a language model may be used to determine the validity of an input source material for further processing, or inclusion in a dataset. For example, a language model may be used to assess the presence (or absence) of a target word in a portion of text or an object in an image, with the text or image being annotated to note such presence (or lack thereof). Alternatively, the determination from the language model may be used to determine whether the source material should be included in a curated dataset, for example and without limitation.

FIG. 5A is a block diagram of an example generative language model system 500 suitable for use in implementing at least some embodiments of the present disclosure. In the example illustrated in FIG. 5A, the generative language model system 500 includes a retrieval augmented generation (RAG) component 592, an input processor 505, a tokenizer 510, an embedding component 520, plug-ins/APIs 595, and a generative language model (LM) 530 (which may include an LLM, a VLM, a multi-modal LM, etc.).

At a high level, the input processor 505 may receive an input 501 comprising text and/or other types of input data (e.g., audio data, video data, image data, sensor data (e.g., LiDAR, RADAR, ultrasonic, etc.), 3D design data, CAD data, universal scene descriptor (USD) data—such as OpenUSD, etc.), depending on the architecture of the generative LM 530 (e.g., LLM/VLM/MMLM/etc.). In some embodiments, the input 501 includes plain text in the form of one or more sentences, paragraphs, and/or documents. Additionally or alternatively, the input 501 may include numerical sequences, precomputed embeddings (e.g., word or sentence embeddings), and/or structured data (e.g., in tabular formats, JSON, or XML). In some implementations in which the generative LM 530 is capable of processing multi-modal inputs, the input 501 may combine text (or may omit text) with image data, audio data, video data, design data, USD data, and/or other types of input data, such as but not limited to those described herein. Taking raw input text as an example, the input processor 505 may prepare raw input text in various ways. For example, the input processor 505 may perform various types of text filtering to remove noise (e.g., special characters, punctuation, HTML tags, stopwords, portions of an image(s), portions of audio, etc.) from relevant textual content. In an example involving stopwords (common words that tend to carry little semantic meaning), the input processor 505 may remove stopwords to reduce noise and focus the generative LM 530 on more meaningful content. The input processor 505 may apply text normalization, for example, by converting all characters to lowercase, removing accents, and/or or handling special cases like contractions or abbreviations to ensure consistency. These are just a few examples, and other types of input processing may be applied.

In some embodiments, a RAG component 592 (which may include one or more RAG models, and/or may be performed using the generative LM 530 itself) may be used to retrieve additional information to be used as part of the input 501 or prompt. RAG may be used to enhance the input to the LLM/VLM/MMLM/etc. with external knowledge, so that answers to specific questions or queries or requests are more relevant—such as in a case where specific knowledge is required. The RAG component 592 may fetch this additional information (e.g., grounding information, such as grounding text/image/video/audio/USD/CAD/etc.) from one or more external sources, which can then be fed to the LLM/VLM/MMLM/etc. along with the prompt to improve accuracy of the responses or outputs of the model.

For example, in some embodiments, the input 501 may be generated using the query or input to the model (e.g., a question, a request, etc.) in addition to data retrieved using the RAG component 592. In some embodiments, the input processor 505 may analyze the input 501 and communicate with the RAG component 592 (or the RAG component 592 may be part of the input processor 505, in embodiments) in order to identify relevant text and/or other data to provide to the generative LM 530 as additional context or sources of information from which to identify the response, answer, or output 590, generally. For example, where the input indicates that the user is interested in a desired tire pressure for a particular make and model of vehicle, the RAG component 592 may retrieve—using a RAG model performing a vector search in an embedding space, for example—the tire pressure information or the text corresponding thereto from a digital (embedded) version of the user manual for that particular vehicle make and model. Similarly, where a user revisits a chatbot related to a particular product offering or service, the RAG component 592 may retrieve a prior stored conversation history—or at least a summary thereof—and include the prior conversation history along with the current ask/request as part of the input 501 to the generative LM 530.

The RAG component 592 may use various RAG techniques. For example, naïve RAG may be used where documents are indexed, chunked, and applied to an embedding model to generate embeddings corresponding to the chunks. A user query may also be applied to the embedding model and/or another embedding model of the RAG component 592 and the embeddings of the chunks along with the embeddings of the query may be compared to identify the most similar/related embeddings to the query, which may be supplied to the generative LM 530 to generate an output.

In some embodiments, more advanced RAG techniques may be used. For example, prior to passing chunks to the embedding model, the chunks may undergo pre-retrieval processes (e.g., routing, rewriting, metadata analysis, expansion, etc.). In addition, prior to generating the final embeddings, post-retrieval processes (e.g., re-ranking, prompt compression, etc.) may be performed on the outputs of the embedding model prior to final embeddings being used as comparison to an input query.

As a further example, modular RAG techniques may be used, such as those that are similar to naïve and/or advanced RAG, but also include features such as hybrid search, recursive retrieval and query engines, StepBack approaches, sub-queries, and hypothetical document embedding.

As another example, Graph RAG may use knowledge graphs as a source of context or factual information. Graph RAG may be implemented using a graph database as a source of contextual information sent to the LLM/VLM/MMLM/etc. Rather than (or in addition to) providing the model with chunks of data extracted from larger sized documents—which may result in a lack of context, factual correctness, language accuracy, etc.—graph RAG may also provide structured entity information to the LLM/VLM/MMLM/etc. by combining the structured entity textual description with its many properties and relationships, allowing for deeper insights by the model. When implementing graph RAG, the systems and methods described herein use a graph as a content store and extract relevant chunks of documents and ask the LLM/VLM/MMLM/etc. to answer using them. The knowledge graph, in such embodiments, may contain relevant textual content and metadata about the knowledge graph as well as be integrated with a vector database. In some embodiments, the graph RAG may use a graph as a subject matter expert, where descriptions of concepts and entities relevant to a query/prompt may be extracted and passed to the model as semantic context. These descriptions may include relationships between the concepts. In other examples, the graph may be used as a database, where part of a query/prompt may be mapped to a graph query, the graph query may be executed, and the LLM/VLM/MMLM/etc. may summarize the results. In such an example, the graph may strore relevant factual information, and a query (natural language query) to graph query tool (NL-to-Graph-query tool) and entity linking may be used. In some embodiments, graph RAG (e.g., using a graph database) may be combined with standard (e.g., vector database) RAG, and/or other RAG types, to benefit from multiple approaches.

In any embodiments, the RAG component 592 may implement a plugin, API, user interface, and/or other functionality to perform RAG. For example, a graph RAG plug-in may be used by the LLM/VLM/MMLM/etc. to run queries against the knowledge graph to extract relevant information for feeding to the model, and a standard or vector RAG plug-in may be used to run queries against a vector database. For example, the graph database may interact with a plug-in's REST interface such that the graph database is decoupled from the vector database and/or the embeddings models.

The tokenizer 510 may segment the (e.g., processed) text data into smaller units (tokens) for subsequent analysis and processing. The tokens may represent individual words, subwords, characters, portions of audio/video/image/etc., depending on the implementation. Word-based tokenization divides the text into individual words, treating each word as a separate token. Subword tokenization breaks down words into smaller meaningful units (e.g., prefixes, suffixes, stems), enabling the generative LM 530 to understand morphological variations and handle out-of-vocabulary words more effectively. Character-based tokenization represents each character as a separate token, enabling the generative LM 530 to process text at a fine-grained level. The choice of tokenization strategy may depend on factors such as the language being processed, the task at hand, and/or characteristics of the training dataset. As such, the tokenizer 510 may convert the (e.g., processed) text into a structured format according to tokenization schema being implemented in the particular embodiment.

The embedding component 520 may use any known embedding technique to transform discrete tokens into (e.g., dense, continuous vector) representations of semantic meaning. For example, the embedding component 520 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise.

In some implementations in which the input 501 includes image data/video data/etc., the input processor 501 may resize the data to a standard size compatible with format of a corresponding input channel and/or may normalize pixel values to a common range (e.g., 0 to 1) to ensure a consistent representation, and the embedding component 520 may encode the image data using any known technique (e.g., using one or more convolutional neural networks (CNNs) to extract visual features). In some implementations in which the input 501 includes audio data, the input processor 501 may resample an audio file to a consistent sampling rate for uniform processing, and the embedding component 520 may use any known technique to extract and encode audio features—such as in the form of a spectrogram (e.g., a mel-spectrogram). In some implementations in which the input 501 includes video data, the input processor 501 may extract frames or apply resizing to extracted frames, and the embedding component 520 may extract features such as optical flow embeddings or video embeddings and/or may encode temporal information or sequences of frames. In some implementations in which the input 501 includes multi-modal data, the embedding component 520 may fuse representations of the different types of data (e.g., text, image, audio, USD, video, design, etc.) using techniques like early fusion (concatenation), late fusion (sequential processing), attention-based fusion (e.g., self-attention, cross-attention), etc.

The generative LM 530 and/or other components of the generative LM system 500 may use different types of neural network architectures depending on the implementation. For example, transformer-based architectures such as those used in models like GPT may be implemented, and may include self-attention mechanisms that weigh the importance of different words or tokens in the input sequence and/or feedforward networks that process the output of the self-attention layers, applying non-linear transformations to the input representations and extracting higher-level features. Some non-limiting example architectures include transformers (e.g., encoder-decoder, decoder only, multi-modal), RNNs, LSTMs, fusion models, diffusion models, cross-modal embedding models that learn joint embedding spaces, graph neural networks (GNNs), hybrid architectures combining different types of architectures adversarial networks like generative adversarial networks or GANs or adversarial autoencoders (AAEs) for joint distribution learning, and others. As such, depending on the implementation and architecture, the embedding component 520 may apply an encoded representation of the input 501 to the generative LM 530, and the generative LM 530 may process the encoded representation of the input 501 to generate an output 590, which may include responsive text and/or other types of data.

As described herein, in some embodiments, the generative LM 530 may be configured to access or use—or capable of accessing or using—plug-ins/APIs 595 (which may include one or more plug-ins, application programming interfaces (APIs), databases, data stores, repositories, etc.). For example, for certain tasks or operations that the generative LM 530 is not ideally suited for, the model may have instructions (e.g., as a result of training, and/or based on instructions in a given prompt, such as those retrieved using the RAG component 592) to access one or more plug-ins/APIs 595 (e.g., 3^{rd} party plugins) for help in processing the current input. In such an example, where at least part of a prompt is related to restaurants or weather, the model may access one or more restaurant or weather plug-ins (e.g., via one or more APIs), send at least a portion of the prompt related to the particular plug-in/API 595 to the plug-in/API 595, the plug-in/API 595 may process the information and return an answer to the generative LM 530, and the generative LM 530 may use the response to generate the output 590. This process may be repeated—e.g., recursively—for any number of iterations and using any number of plug-ins/APIs 595 until an output 590 that addresses each ask/question/request/process/operation/etc. from the input 501 can be generated. As such, the model(s) may not only rely on its own knowledge from training on a large dataset(s) and/or from data retrieved using the RAG component 592, but also on the expertise or optimized nature of one or more external resources—such as the plug-ins/APIs 595.

FIG. 5B is a block diagram of an example implementation in which the generative LM 530 includes a transformer encoder-decoder. For example, assume input text such as “Who discovered gravity” is tokenized (e.g., by the tokenizer 510 of FIG. 5A) into tokens such as words, and each token is encoded (e.g., by the embedding component 520 of FIG. 95A) into a corresponding embedding (e.g., of size 512). Since these token embeddings typically do not represent the position of the token in the input sequence, any known technique may be used to add a positional encoding to each token embedding to encode the sequential relationships and context of the tokens in the input sequence. As such, the (e.g., resulting) embeddings may be applied to one or more encoder(s) 535 of the generative LM 530.

In an example implementation, the encoder(s) 535 forms an encoder stack, where each encoder includes a self-attention layer and a feedforward network. In an example transformer architecture, each token (e.g., word) flows through a separate path. As such, each encoder may accept a sequence of vectors, passing each vector through the self-attention layer, then the feedforward network, and then upwards to the next encoder in the stack. Any known self-attention technique may be used. For example, to calculate a self-attention score for each token (word), a query vector, a key vector, and a value vector may be created for each token, a self-attention score may be calculated for pairs of tokens by taking the dot product of the query vector with the corresponding key vectors, normalizing the resulting scores, multiplying by corresponding value vectors, and summing weighted value vectors. The encoder may apply multi-headed attention in which the attention mechanism is applied multiple times in parallel with different learned weight matrices. Any number of encoders may be cascaded to generate a context vector encoding the input. An attention projection layer 540 may convert the context vector into attention vectors (keys and values) for the decoder(s) 545.

In an example implementation, the decoder(s) 545 form a decoder stack, where each decoder includes a self-attention layer, an encoder-decoder self-attention layer that uses the attention vectors (keys and values) from the encoder to focus on relevant parts of the input sequence, and a feedforward network. As with the encoder(s) 535, in an example transformer architecture, each token (e.g., word) flows through a separate path in the decoder(s) 545. During a first pass, the decoder(s) 545, a classifier 550, and a generation mechanism 555 may generate a first token, and the generation mechanism 555 may apply the generated token as an input during a second pass. The process may repeat in a loop, successively generating and adding tokens (e.g., words) to the output from the preceding pass and applying the token embeddings of the composite sequence with positional encodings as an input to the decoder(s) 545 during a subsequent pass, sequentially generating one token at a time (known as auto-regression) until predicting a symbol or token that represents the end of the response. Within each decoder, the self-attention layer is typically constrained to attend only to preceding positions in the output sequence by applying a masking technique (e.g., setting future positions to negative infinity) before the softmax operation. In an example implementation, the encoder-decoder attention layer operates similarly to the (e.g., multi-headed) self-attention in the encoder(s) 535, except that it creates its queries from the layer below it and takes the keys and values (e.g., matrix) from the output of the encoder(s) 535.

As such, the decoder(s) 545 may output some decoded (e.g., vector) representation of the input being applied during a particular pass. The classifier 550 may include a multi-class classifier comprising one or more neural network layers that project the decoded (e.g., vector) representation into a corresponding dimensionality (e.g., one dimension for each supported word or token in the output vocabulary) and a softmax operation that converts logits to probabilities. As such, the generation mechanism 555 may select or sample a word or token based on a corresponding predicted probability (e.g., select the word with the highest predicted probability) and append it to the output from a previous pass, generating each word or token sequentially. The generation mechanism 555 may repeat the process, triggering successive decoder inputs and corresponding predictions until selecting or sampling a symbol or token that represents the end of the response, at which point, the generation mechanism 555 may output the generated response.

FIG. 5C is a block diagram of an example implementation in which the generative LM 530 includes a decoder-only transformer architecture. For example, the decoder(s) 560 of FIG. 5C may operate similarly as the decoder(s) 545 of FIG. 5B except each of the decoder(s) 560 of FIG. 5C omits the encoder-decoder self-attention layer (since there is no encoder in this implementation). As such, the decoder(s) 560 may form a decoder stack, where each decoder includes a self-attention layer and a feedforward network. Furthermore, instead of encoding the input sequence, a symbol or token representing the end of the input sequence (or the beginning of the output sequence) may be appended to the input sequence, and the resulting sequence (e.g., corresponding embeddings with positional encodings) may be applied to the decoder(s) 560. As with the decoder(s) 545 of FIG. 5B, each token (e.g., word) may flow through a separate path in the decoder(s) 560, and the decoder(s) 560, a classifier 565, and a generation mechanism 570 may use auto-regression to sequentially generate one token at a time until predicting a symbol or token that represents the end of the response. The classifier 565 and the generation mechanism 570 may operate similarly as the classifier 550 and the generation mechanism 555 of FIG. 5B, with the generation mechanism 570 selecting or sampling each successive output token based on a corresponding predicted probability and appending it to the output from a previous pass, generating each token sequentially until selecting or sampling a symbol or token that represents the end of the response. These and other architectures described herein are meant simply as examples, and other suitable architectures may be implemented within the scope of the present disclosure.

### Machine Learning

Deep neural networks (DNNs) and other machine learning models have been used for diverse use cases, from self-driving cars to faster drug development, from automatic image captioning in online image databases to smart real-time language translation in video chat applications. Deep learning is a technique that models the neural learning process of the human brain, continually learning, continually getting smarter, and delivering more accurate results more quickly over time. A child is initially taught by an adult to correctly identify and classify various shapes, eventually being able to identify shapes without any coaching. Similarly, a deep learning or neural learning system may be trained in object recognition and classification to identify objects and classify those objects.

At the simplest level, neurons in the human brain look at various inputs that are received, importance levels are assigned to each of these inputs, and output is passed on to other neurons to act upon. An artificial neuron or perceptron is the most basic model of a neural network. In one example, a perceptron may receive one or more inputs that represent various features of an object that the perceptron is being trained to recognize and classify, and each of these features may be assigned a certain weight based on the importance of that feature in defining the shape of an object.

A DNN model typically includes multiple layers of many connected nodes (e.g., perceptrons, Boltzmann machines, radial basis functions, convolutional layers, etc.) that can be trained with enormous amounts of input data to quickly solve complex problems with high accuracy. In one example, a first layer of the DNN model breaks down an input image of an automobile into various sections and looks for basic patterns such as lines and angles. The second layer may assemble the lines to look for higher level patterns such as wheels, windshields, and mirrors. The next layer may identify a type of vehicle, and the final few layers may generate a label for the input image, identifying the model of a specific automobile brand.

Once the DNN is trained, it may be deployed and used to identify and classify objects or patterns in a process known as inference. Examples of inference (the process through which a DNN extracts useful information from a given input) include identifying handwritten numbers on checks deposited into ATM machines, identifying images of friends in photos, delivering movie recommendations to over fifty million users, identifying and classifying different types of automobiles, pedestrians, and road hazards in driverless cars, or translating human speech in real-time.

During training, data flows through the DNN in a forward propagation phase until a prediction is produced that indicates a label corresponding to the input. If the neural network does not correctly label the input, then errors between the correct label and the predicted label may be analyzed, and the weights may be adjusted for each feature during a backward propagation phase until the DNN correctly labels the input and other inputs in a training dataset. Training complex neural networks requires massive amounts of parallel computing performance, including floating-point multiplications and additions that may be supported by a parallel processing unit.

Whether during training, testing, or inference, neural networks rely heavily on matrix math operations, and complex multi-layered networks require tremendous amounts of floating-point performance and bandwidth for both efficiency and speed. In some embodiments (e.g., with thousands of processing cores, optimized for matrix math operations, delivering tens to hundreds of TFLOPS of performance), the parallel processing unit may be a computing platform capable of supporting deep neural network-based artificial intelligence and machine learning applications.

In some examples, the machine learning model(s) (e.g., deep neural networks, language models, LLMs, VLMs, multi-modal language models, perception models, tracking models, fusion models, transformer models, diffusion models, encoder-only models, decoder-only models, encoder-decoder models, neural rendering field (NERF) models, etc.) described herein may be packaged as a microservice—such an inference microservice (e.g., NVIDIA NIMs)—which may include a container (e.g., an operating system (OS)-level virtualization package) that may include an application programming interface (API) layer, a server layer, a runtime layer, and/or at least one model “engine.” For example, the inference microservice may include the container itself and the model(s) (e.g., weights and biases). In some instances, such as where the machine learning model(s) is small enough (e.g., has a small enough number of parameters), the model(s) may be included within the container itself. In other examples—such as where the model(s) is large—the model(s) may be hosted/stored in the cloud (e.g., in a data center) and/or may be hosted on-premises and/or at the edge (e.g., on a local server or computing device, but outside of the container). In such embodiments, the model(s) may be accessible via one or more APIs—such as REST APIs. As such, and in some embodiments, the machine learning model(s) described herein may be deployed as an inference microservice to accelerate deployment of a model(s) on any cloud, data center, or edge computing system, while ensuring the data is secure. For example, the inference microservice may include one or more APIs, a pre-configured container for simplified deployment, an optimized inference engine (e.g., built using a standardized AI model deployment an execution software, such as NVIDIA's Triton Inference Server, and/or one or more APIs for high performance deep learning inference, which may include an inference runtime and model optimizations that deliver low latency and high throughput for production applications—such as NVIDIA's TensorRT), and/or enterprise management data for telemetry (e.g., including identity, metrics, health checks, and/or monitoring).

The machine learning model(s) described herein may be included as part of the microservice along with an accelerated infrastructure with the ability to deploy with a single command and/or orchestrate and auto-scale with a container orchestration system on accelerated infrastructure (e.g., on a single device up to data center scale). As such, the inference microservice may include the machine learning model(s) (e.g., that has been optimized for high performance inference), an inference runtime software to execute the machine learning model(s) and provide outputs/responses to inputs (e.g., user queries, prompts, etc.), and enterprise management software to provide health checks, identity, and/or other monitoring. In some embodiments, the inference microservice may include software to perform in-place replacement and/or updating to the machine learning model(s). When replacing or updating, the software that performs the replacement/updating may maintain user configurations of the inference runtime software and enterprise management software.

Although examples may be described herein with respect to using machine learning models, such as neural networks, this is not intended to be limiting. For example, and without limitation, any of the various machine learning models and/or neural networks described herein may include any type of machine learning model, such as a machine learning model(s) using linear regression, logistic regression, decision trees, support vector machines (SVM), Naïve Bayes, k-nearest neighbor (Knn), K means clustering, random forest, dimensionality reduction algorithms, gradient boosting algorithms, neural networks (e.g., auto-encoder neural networks, artificial neural networks (ANNs), convolutional neural networks (CNNs), recurrent neural networks (RNNs), perceptrons, Long/Short Term Memory (LSTM) networks, multi-layer perceptron (MLP) networks, deep stacking networks (DSNs), generative pre-training (GPT) models or networks, feed forward networks, radial basis function ANNs, self-organizing maps (SOMs), Kohonen maps, Hopfield networks, Boltzmann machine, deep belief neural networks, deconvolutional neural networks, generative adversarial networks (GANs), liquid state machines, modular neural networks, liquid state machines, sequence-to-sequence models, networks using transformer architectures, diffusion models (e.g., diffusion probabilistic models, score-based generative models, etc.), neural rendering field (NeRF) models, Kolmogorov-Arnold networks (KANs), models with encoder-only architectures, models with decoder-only architectures, models with encoder-decoder architectures, generative machine learning models, language models, large language models (LLMs), vision language models (VLMs), multi-modal language models (MMLMs), etc.), and/or other types of machine learning models.

### Example Computing Device

FIG. 6 is a block diagram of an example computing device(s) 600 suitable for use in implementing some embodiments of the present disclosure. Computing device 600 may include an interconnect system 602 that directly or indirectly couples the following devices: memory 604, one or more central processing units (CPUs) 606, one or more graphics processing units (GPUs) 608, a communication interface 610, input/output (I/O) ports 612, input/output components 614, a power supply 616, one or more presentation components 618 (e.g., display(s)), and one or more logic units 620. In at least one embodiment, the computing device(s) 600 may comprise one or more virtual machines (VMs), and/or any of the components thereof may comprise virtual components (e.g., virtual hardware components). For non-limiting examples, one or more of the GPUs 608 may comprise one or more vGPUs, one or more of the CPUs 606 may comprise one or more vCPUs, and/or one or more of the logic units 620 may comprise one or more virtual logic units. As such, a computing device(s) 600 may include discrete components (e.g., a full GPU dedicated to the computing device 600), virtual components (e.g., a portion of a GPU dedicated to the computing device 600), or a combination thereof.

Although the various blocks of FIG. 6 are shown as connected via the interconnect system 602 with lines, this is not intended to be limiting and is for clarity only. For example, in some embodiments, a presentation component 618, such as a display device, may be considered an I/O component 614 (e.g., if the display is a touch screen). As another example, the CPUs 606 and/or GPUs 608 may include memory (e.g., the memory 604 may be representative of a storage device in addition to the memory of the GPUs 608, the CPUs 606, and/or other components). As such, the computing device of FIG. 6 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” and/or other device or system types, as all are contemplated within the scope of the computing device of FIG. 6.

The interconnect system 602 may represent one or more links or busses, such as an address bus, a data bus, a control bus, or a combination thereof. The interconnect system 602 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 606 may be directly connected to the memory 604. Further, the CPU 606 may be directly connected to the GPU 608. Where there is direct, or point-to-point connection between components, the interconnect system 602 may include a PCIe link to carry out the connection. In these examples, a PCI bus need not be included in the computing device 600.

The memory 604 may include any of a variety of computer-readable media. The computer-readable media may be any available media that may be accessed by the computing device 600. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 604 may store computer-readable instructions (e.g., that represent a program(s) and/or a program element(s), such as an operating system). Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which may be used to store the desired information and which may be accessed by computing device 600. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 606 may be configured to execute at least some of the computer-readable instructions to control one or more components of the computing device 600 to perform one or more of the methods and/or processes described herein. The CPU(s) 606 may each include one or more cores (e.g., one, two, four, eight, twenty-eight, seventy-two, etc.) that are capable of handling a multitude of software threads simultaneously. The CPU(s) 606 may include any type of processor, and may include different types of processors depending on the type of computing device 600 implemented (e.g., processors with fewer cores for mobile devices and processors with more cores for servers). For example, depending on the type of computing device 600, the processor may be an Advanced RISC Machines (ARM) processor implemented using Reduced Instruction Set Computing (RISC) or an x86 processor implemented using Complex Instruction Set Computing (CISC). The computing device 600 may include one or more CPUs 606 in addition to one or more microprocessors or supplementary co-processors, such as math co-processors.

In addition to or alternatively from the CPU(s) 606, the GPU(s) 608 may be configured to execute at least some of the computer-readable instructions to control one or more components of the computing device 600 to perform one or more of the methods and/or processes described herein. One or more of the GPU(s) 608 may be an integrated GPU (e.g., with one or more of the CPU(s) 606) and/or one or more of the GPU(s) 608 may be a discrete GPU. In embodiments, one or more of the GPU(s) 608 may be a coprocessor of one or more of the CPU(s) 606. The GPU(s) 608 may be used by the computing device 600 to render graphics (e.g., 3D graphics) or perform general purpose computations. For example, the GPU(s) 608 may be used for General-Purpose computing on GPUs (GPGPU). The GPU(s) 608 may include hundreds or thousands of cores that are capable of handling hundreds or thousands of software threads simultaneously. The GPU(s) 608 may generate pixel data for output images in response to rendering commands (e.g., rendering commands from the CPU(s) 606 received via a host interface). The GPU(s) 608 may include graphics memory, such as display memory, for storing pixel data or any other suitable data, such as GPGPU data. The display memory may be included as part of the memory 604. The GPU(s) 608 may include two or more GPUs operating in parallel (e.g., via a link). The link may directly connect the GPUs (e.g., using NVLINK) or may connect the GPUs through a switch (e.g., using NVSwitch). When combined together, each GPU 608 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 606 and/or the GPU(s) 608, the logic unit(s) 620 may be configured to execute at least some of the computer-readable instructions to control one or more components of the computing device 600 to perform one or more of the methods and/or processes described herein. In embodiments, the CPU(s) 606, the GPU(s) 608, and/or the logic unit(s) 620 may discretely or jointly perform any combination of the methods, processes and/or portions thereof. One or more of the logic units 620 may be part of and/or integrated in one or more of the CPU(s) 606 and/or the GPU(s) 608 and/or one or more of the logic units 620 may be discrete components or otherwise external to the CPU(s) 606 and/or the GPU(s) 608. In embodiments, one or more of the logic units 620 may be a coprocessor of one or more of the CPU(s) 606 and/or one or more of the GPU(s) 608.

Examples of the logic unit(s) 620 include one or more processing cores and/or components thereof, such as Data Processing Units (DPUs), Tensor Cores (TCs), Tensor Processing Units (TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Programmable Vision Accelerator (PVAs)—which may include one or more direct memory access (DMA) systems, one or more vision or vector processing units (VPUs), one or more pixel processing engines (PPEs)—e.g., including a 2D array of processing elements that each communicate north, south, east, and west with one or more other processing elements in the array, one or more decoupled accelerators or units (e.g., decoupled lookup table (DLUT) accelerators or units), etc., Vision Processing Units (VPUs), Optical Flow Accelerators (OFAs), Field Programmable Gate Arrays (FPGAs), Neuromorphic Chips, Quantum Processing Units (QPUs), Associative Process Units (APUs), Arithmetic-Logic Units (ALUs), Application-Specific Integrated Circuits (ASICs), Floating Point Units (FPUs), input/output (I/O) elements, peripheral component interconnect (PCI) or peripheral component interconnect express (PCIe) elements, and/or the like.

The communication interface 610 may include one or more receivers, transmitters, and/or transceivers that allow the computing device 600 to communicate with other computing devices via an electronic communication network, included wired and/or wireless communications. The communication interface 610 may include components and functionality to allow communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet. In one or more embodiments, logic unit(s) 620 and/or communication interface 610 may include one or more data processing units (DPUs) to transmit data received over a network and/or through interconnect system 602 directly to (e.g., a memory of) one or more GPU(s) 608.

The I/O ports 612 may allow the computing device 600 to be logically coupled to other devices including the I/O components 614, the presentation component(s) 618, and/or other components, some of which may be built in to (e.g., integrated in) the computing device 600. Illustrative I/O components 614 include a microphone, mouse, keyboard, joystick, game pad, game controller, satellite dish, scanner, printer, wireless device, etc. The I/O components 614 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing device 600. The computing device 600 may be include depth cameras, such as stereoscopic camera systems, infrared camera systems, RGB camera systems, touchscreen technology, and combinations of these, for gesture detection and recognition. Additionally, the computing device 600 may include accelerometers or gyroscopes (e.g., as part of an inertia measurement unit (IMU)) that allow detection of motion. In some examples, the output of the accelerometers or gyroscopes may be used by the computing device 600 to render immersive augmented reality or virtual reality.

The power supply 616 may include a hard-wired power supply, a battery power supply, or a combination thereof. The power supply 616 may provide power to the computing device 600 to allow the components of the computing device 600 to operate.

The presentation component(s) 618 may include a display (e.g., a monitor, a touch screen, a television screen, a heads-up-display (HUD), other display types, or a combination thereof), speakers, and/or other presentation components. The presentation component(s) 618 may receive data from other components (e.g., the GPU(s) 608, the CPU(s) 606, DPUs, etc.), and output the data (e.g., as an image, video, sound, etc.).

### Example Data Center

FIG. 7 illustrates an example data center 700 that may be used in at least one embodiments of the present disclosure. The data center 700 may include a data center infrastructure layer 710, a framework layer 720, a software layer 730, and/or an application layer 740.

As shown in FIG. 7, the data center infrastructure layer 710 may include a resource orchestrator 712, grouped computing resources 714, and node computing resources (“node C.R.s”) 716(1)-716(N), where “N” represents any whole, positive integer. In at least one embodiment, node C.R.s 716(1)-716(N) may include, but are not limited to, any number of central processing units (CPUs) or other processors (including DPUs, accelerators, field programmable gate arrays (FPGAs), graphics processors or graphics processing units (GPUs), etc.), memory devices (e.g., dynamic read-only memory), storage devices (e.g., solid state or disk drives), network input/output (NW I/O) devices, network switches, virtual machines (VMs), power modules, and/or cooling modules, etc. In some embodiments, one or more node C.R.s from among node C.R.s 716(1)-716(N) may correspond to a server having one or more of the above-mentioned computing resources. In addition, in some embodiments, the node C.R.s 716(1)-7161(N) may include one or more virtual components, such as vGPUs, vCPUs, and/or the like, and/or one or more of the node C.R.s 716(1)-716(N) may correspond to a virtual machine (VM).

In at least one embodiment, grouped computing resources 714 may include separate groupings of node C.R.s 716 housed within one or more racks (not shown), or many racks housed in data centers at various geographical locations (also not shown). Separate groupings of node C.R.s 716 within grouped computing resources 714 may include grouped compute, network, memory or storage resources that may be configured or allocated to support one or more workloads. In at least one embodiment, several node C.R.s 716 including CPUs, GPUs, DPUs, and/or other processors may be grouped within one or more racks to provide compute resources to support one or more workloads. The one or more racks may also include any number of power modules, cooling modules, and/or network switches, in any combination.

The resource orchestrator 712 may configure or otherwise control one or more node C.R.s 716(1)-716(N) and/or grouped computing resources 714. In at least one embodiment, resource orchestrator 712 may include a software design infrastructure (SDI) management entity for the data center 700. The resource orchestrator 712 may include hardware, software, or some combination thereof.

In at least one embodiment, as shown in FIG. 7, framework layer 720 may include a job scheduler 728, a configuration manager 734, a resource manager 736, and/or a distributed file system 738. The framework layer 720 may include a framework to support software 732 of software layer 730 and/or one or more application(s) 742 of application layer 740. The software 732 or application(s) 742 may respectively include web-based service software or applications, such as those provided by Amazon Web Services, Google Cloud and Microsoft Azure. The framework layer 720 may be, but is not limited to, a type of free and open-source software web application framework such as Apache Spark™ (hereinafter “Spark”) that may use distributed file system 738 for large-scale data processing (e.g., “big data”). In at least one embodiment, job scheduler 728 may include a Spark driver to facilitate scheduling of workloads supported by various layers of data center 700. The configuration manager 734 may be capable of configuring different layers such as software layer 730 and framework layer 720 including Spark and distributed file system 738 for supporting large-scale data processing. The resource manager 736 may be capable of managing clustered or grouped computing resources mapped to or allocated for support of distributed file system 738 and job scheduler 728. In at least one embodiment, clustered or grouped computing resources may include grouped computing resource 714 at data center infrastructure layer 710. The resource manager 736 may coordinate with resource orchestrator 712 to manage these mapped or allocated computing resources.

In at least one embodiment, software 732 included in software layer 730 may include software used by at least portions of node C.R.s 716(1)-716(N), grouped computing resources 714, and/or distributed file system 738 of framework layer 720. One or more types of software may include, but are not limited to, Internet web page search software, e-mail virus scan software, database software, and streaming video content software.

In at least one embodiment, application(s) 742 included in application layer 740 may include one or more types of applications used by at least portions of node C.R.s 716(1)-716(N), grouped computing resources 714, and/or distributed file system 738 of framework layer 720. One or more types of applications may include, but are not limited to, any number of a genomics application, a cognitive compute, and a machine learning application, including training or inferencing software, machine learning framework software (e.g., PyTorch, TensorFlow, Caffe, etc.), and/or other machine learning applications used in conjunction with one or more embodiments.

In at least one embodiment, any of configuration manager 734, resource manager 736, and resource orchestrator 712 may implement any number and type of self-modifying actions based on any amount and type of data acquired in any technically feasible fashion. Self-modifying actions may relieve a data center operator of data center 700 from making possibly bad configuration decisions and possibly avoiding underutilized and/or poor performing portions of a data center.

The data center 700 may include tools, services, software or other resources to train one or more machine learning models or predict or infer information using one or more machine learning models according to one or more embodiments described herein. For example, a machine learning model(s) may be trained by calculating weight parameters according to a neural network architecture using software and/or computing resources described above with respect to the data center 700. In at least one embodiment, trained or deployed machine learning models corresponding to one or more neural networks may be used to infer or predict information using resources described above with respect to the data center 700 by using weight parameters calculated through one or more training techniques, such as but not limited to those described herein.

In at least one embodiment, the data center 700 may use CPUs, application-specific integrated circuits (ASICs), GPUs, FPGAs, and/or other hardware (or virtual compute resources corresponding thereto) to perform training and/or inferencing using above-described resources. Moreover, one or more software and/or hardware resources described above may be configured as a service to allow users to train or performing inferencing of information, such as image recognition, speech recognition, or other artificial intelligence services.

### Example Network Environments

Network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing device(s) 600 of FIG. 6—e.g., each device may include similar components, features, and/or functionality of the computing device(s) 600. In addition, where backend devices (e.g., servers, NAS, etc.) are implemented, the backend devices may be included as part of a data center 700, an example of which is described in more detail herein with respect to FIG. 7.

Components of a network environment may communicate with each other via a network(s), which may be wired, wireless, or both. The network may include multiple networks, or a network of networks. By way of example, the network may include one or more Wide Area Networks (WANs), one or more Local Area Networks (LANs), one or more public networks such as the Internet and/or a public switched telephone network (PSTN), and/or one or more private networks. Where the network includes a wireless telecommunications network, components such as a base station, a communications tower, or even access points (as well as other components) may provide wireless connectivity.

Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may include a framework layer, a job scheduler, a resource manager, and a distributed file system implemented on one or more of servers, which may include one or more core network servers and/or edge servers. A framework layer may include a framework to support software of a software layer and/or one or more application(s) of an application layer. The software or application(s) may respectively include web-based service software or applications. In embodiments, one or more of the client devices may use the web-based service software or applications (e.g., by accessing the service software and/or applications via one or more application programming interfaces (APIs)). The framework layer may be, but is not limited to, a type of free and open-source software web application framework such as that may use a distributed file system for large-scale data processing (e.g., “big data”).

A cloud-based network environment may provide cloud computing and/or cloud storage that carries out any combination of computing and/or data storage functions described herein (or one or more portions thereof). Any of these various functions may be distributed over multiple locations from central or core servers (e.g., of one or more data centers that may be distributed across a state, a region, a country, the globe, etc.). If a connection to a user (e.g., a client device) is relatively close to an edge server(s), a core server(s) may designate at least a portion of the functionality to the edge server(s). A cloud-based network environment may be private (e.g., limited to a single organization), may be public (e.g., available to many organizations), and/or a combination thereof (e.g., a hybrid cloud environment).

The client device(s) may include at least some of the components, features, and functionality of the example computing device(s) 600 described herein with respect to FIG. 6. By way of example and not limitation, a client device may be embodied as a Personal Computer (PC), a laptop computer, a mobile device, a smartphone, a tablet computer, a smart watch, a wearable computer, a Personal Digital Assistant (PDA), an MP3 player, a virtual reality headset, a Global Positioning System (GPS) or device, a video player, a video camera, a surveillance device or system, a vehicle, a boat, a flying vessel, a virtual machine, a drone, a robot, a handheld communications device, a hospital device, a gaming device or system, an entertainment system, a vehicle computer system, an embedded system controller, a remote control, an appliance, a consumer electronic device, a workstation, an edge device, any combination of these delineated devices, or any other suitable device.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular abstract data types. The disclosure may be practiced in a variety of system configurations, including hand-held devices, consumer electronics, general-purpose computers, more specialty computing devices, etc. The disclosure may also be practiced in distributed computing environments where tasks are performed by remote-processing devices that are linked through a communications network.

### Example Literal Support

The disclosure of this application also includes the following numbered clauses:

Clause 1. One or more processors comprising processing circuitry to generate, based at least on applying a representation of free-form input text to a diffusion model, a latent embedding representative of the free-form input text.

Clause 2. The one or more processors of clause 1, wherein the processing circuitry is further to generate simulated three-dimensional (3D) image data based at least on applying the latent embedding to a visual decoder of a 3D compression network.

Clause 3. The one or more processors of clause 1 or 2, wherein the processing circuitry is further to include the simulated 3D image data in one or more datasets.

Clause 4. The one or more processors of clause 1, 2 or 3, wherein the processing circuitry is further to update the diffusion model with a visual encoder of the 3D compression network.

Clause 5. The one or more processors of clause 1, 2 or 3, wherein the processing circuitry is further to update the diffusion model using input data comprising one or more text descriptions of 3D image training data, the one or more text descriptions generated using one or more language models.

Clause 6. The one or more processors of clause 5, wherein the 3D image training data comprises 3D medical image training data, and the processing circuitry is further to generate the one or more text descriptions of the 3D medical image training data based on at least one of: one or more prompts that provide the one or more language models with at least a portion of one or more corresponding radiology reports; one or more prompts that instruct the one or more language models to generate free-form text describing one or more corresponding radiology reports; one or more prompts that instruct the one or more language models to rephrase at least a portion of one or more radiology reports; or one or more prompts that provide the one or more language models with one or more lists of one or more organs detected based at least on applying a representation of the 3D medical image training data to one or more neural networks.

Clause 7. The one or more processors of clause 1, 2 or 3, wherein the processing circuitry is further to update the diffusion model using one or more training pairs comprising one or more input text descriptions sampled from one or more fields of one or more augmented radiology reports.

Clause 8. The one or more processors of clause 1, 2 or 3, wherein the simulated 3D image data comprises at least one of one or more computed tomography (CT) scans, one or more magnetic resonance imaging (MRI) scans, or one or more positron emission tomography (PET) scans.

Clause 9. The one or more processors of clause 1, 2 or 3, wherein the processing circuitry is further to update one or more machine learning models using the simulated 3D image data in the one or more datasets.

Clause 10. The one or more processors of clause 1, 2 or 3, wherein the one or more processors are comprised in at least one of: a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; or a system implemented at least partially using cloud computing resources.

Clause 11. A method comprising generating simulated three-dimensional (3D) image data based at least on applying a representation of one or more input text descriptions to a neural network comprising a diffusion model and a visual decoder of a 3D compression network.

Clause 12. The method of clause 11, further comprising including the simulated 3D image data in one or more datasets.

Clause 13. The method of clause 11 or 12, further comprising updating the diffusion model with a visual encoder of the 3D compression network.

Clause 14. The method of clause 11 or 12, wherein the 3D image training data comprises 3D medical image training data, the method further comprising updating the diffusion model using input training data comprising one or more text descriptions of the 3D medical image training data, the one or more text descriptions generated using one or more language models.

Clause 15. The method of clause 11 or 12, further comprising training the diffusion model using one or more training pairs comprising one or more text descriptions sampled from fields of augmented radiology reports.

Clause 16. The method of clause 11 or 12, wherein the simulated 3D image data comprises at least one of: one or more computed tomography (CT) scans, one or more magnetic resonance imaging (MRI) scans, or one or more positron emission tomography (PET) scans.

Clause 17. The method of clause 11 or 12, wherein the method is performed by at least one of: a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; or a system implemented at least partially using cloud computing resources. 15. A system comprising: one or more processors to perform one or more updating iterations of one or more machine learning models based at least on training data comprising simulated three-dimensional (3D) image data, the simulated 3D image data generated based at least on applying a representation of an input text description to a neural network comprising a diffusion model and a visual decoder of a 3D compression network.

Clause 18. The system of clause 15, wherein at least one of the one or more machine learning models or at least a portion of the neural network is implemented in at least one processing node of a plurality of processing nodes of a data center and accessible to one or more remote clients via at least one of an application programming interface (API), or an application plug-in.

Clause 19. The system of clause 15, wherein the diffusion model is updated with a visual encoder of the 3D compression network.

Clause 20. The system of clause 15, wherein the 3D image training data comprises 3D medical image training data, and the diffusion model is updated using input training data comprising one or more text descriptions of the 3D medical image training data, the one or more text descriptions generated using one or more language models.

Clause 21. The system of clause 15, wherein the simulated 3D image data comprises at least one of: one or more computed tomography (CT) scans, one or more magnetic resonance imaging (MRI) scans, or one or more positron emission tomography (PET) scans.

Clause 22. The system of clause 15, wherein the system is comprised in at least one of: a system for performing simulation operations; a system for performing digital twin operations; a system for performing light transport simulation; a system for performing collaborative content creation for 3D assets; a system for performing deep learning operations; a system for performing remote operations; a system for performing real-time streaming; a system for generating or presenting one or more of augmented reality content, virtual reality content, or mixed reality content; a system implemented using an edge device; a system for performing conversational AI operations; a system implementing one or more language models; a system implementing one or more large language models (LLMs); a system implementing one or more vision language models (VLMs); a system implementing one or more multi-modal language models; a system for generating synthetic data; a system for generating synthetic data using AI; a system for performing one or more generative AI operations; a system incorporating one or more virtual machines (VMs); a system implemented at least partially in a data center; or a system implemented at least partially using cloud computing resources.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

What is claimed is:

1. One or more processors comprising processing circuitry to:
generate, based at least on applying a representation of free-form input text to a diffusion model, a latent embedding representative of the free-form input text;
generate simulated three-dimensional (3D) image data based at least on applying the latent embedding to a visual decoder of a 3D compression network; and
include the simulated 3D image data in one or more datasets.

2. The one or more processors of claim 1, wherein the processing circuitry is further to update the diffusion model with a visual encoder of the 3D compression network.

3. The one or more processors of claim 1, wherein the processing circuitry is further to update the diffusion model using input data comprising one or more text descriptions of 3D image training data, the one or more text descriptions generated using one or more language models.

4. The one or more processors of claim 3, wherein the 3D image training data comprises 3D medical image training data, and the processing circuitry is further to generate the one or more text descriptions of the 3D medical image training data based on at least one of:
one or more prompts that provide the one or more language models with at least a portion of one or more corresponding radiology reports;
one or more prompts that instruct the one or more language models to generate free-form text describing one or more corresponding radiology reports;
one or more prompts that instruct the one or more language models to rephrase at least a portion of one or more radiology reports; or
one or more prompts that provide the one or more language models with one or more lists of one or more organs detected based at least on applying a representation of the 3D medical image training data to one or more neural networks.

5. The one or more processors of claim 1, wherein the processing circuitry is further to update the diffusion model using one or more training pairs comprising one or more input text descriptions sampled from one or more fields of one or more augmented radiology reports.

6. The one or more processors of claim 1, wherein the simulated 3D image data comprises at least one of one or more computed tomography (CT) scans, one or more magnetic resonance imaging (MRI) scans, or one or more positron emission tomography (PET) scans.

7. The one or more processors of claim 1, wherein the processing circuitry is further to update one or more machine learning models using the simulated 3D image data in the one or more datasets.

8. The one or more processors of claim 1, wherein the one or more processors are comprised in at least one of:
a system for performing simulation operations;
a system for performing digital twin operations;
a system for performing light transport simulation;
a system for performing collaborative content creation for 3D assets;
a system for performing deep learning operations;
a system for performing remote operations;
a system for performing real-time streaming;
a system for generating or presenting one or more of augmented reality content, virtual reality content, or mixed reality content;
a system implemented using an edge device;
a system for performing conversational AI operations;
a system implementing one or more language models;
a system implementing one or more large language models (LLMs);
a system implementing one or more vision language models (VLMs);
a system implementing one or more multi-modal language models;
a system for generating synthetic data;
a system for generating synthetic data using AI;
a system for performing one or more generative AI operations;
a system incorporating one or more virtual machines (VMs);
a system implemented at least partially in a data center; or
a system implemented at least partially using cloud computing resources.

9. A method comprising:
generating simulated three-dimensional (3D) image data based at least on applying a representation of one or more input text descriptions to a neural network comprising a diffusion model and a visual decoder of a 3D compression network; and
including the simulated 3D image data in one or more datasets.

10. The method of claim 9, further comprising updating the diffusion model with a visual encoder of the 3D compression network.

11. The method of claim 9, wherein the 3D image training data comprises 3D medical image training data, the method further comprising updating the diffusion model using input training data comprising one or more text descriptions of the 3D medical image training data, the one or more text descriptions generated using one or more language models.

12. The method of claim 9, further comprising training the diffusion model using one or more training pairs comprising one or more text descriptions sampled from fields of augmented radiology reports.

13. The method of claim 9, wherein the simulated 3D image data comprises at least one of: one or more computed tomography (CT) scans, one or more magnetic resonance imaging (MRI) scans, or one or more positron emission tomography (PET) scans.

14. The method of claim 9, wherein the method is performed by at least one of:
a system for performing simulation operations;
a system for performing digital twin operations;
a system for performing light transport simulation;
a system for performing collaborative content creation for 3D assets;
a system for performing deep learning operations;
a system for performing remote operations;
a system for performing real-time streaming;
a system for generating or presenting one or more of augmented reality content, virtual reality content, or mixed reality content;
a system implemented using an edge device;
a system for performing conversational AI operations;
a system implementing one or more language models;
a system implementing one or more large language models (LLMs);
a system implementing one or more vision language models (VLMs);
a system implementing one or more multi-modal language models;
a system for generating synthetic data;
a system for generating synthetic data using AI;
a system for performing one or more generative AI operations;
a system incorporating one or more virtual machines (VMs);
a system implemented at least partially in a data center; or
a system implemented at least partially using cloud computing resources.

15. A system comprising:
one or more processors to perform one or more updating iterations of one or more machine learning models based at least on training data comprising simulated three-dimensional (3D) image data, the simulated 3D image data generated based at least on applying a representation of an input text description to a neural network comprising a diffusion model and a visual decoder of a 3D compression network.

16. The system of claim 15, wherein at least one of the one or more machine learning models or at least a portion of the neural network is implemented in at least one processing node of a plurality of processing nodes of a data center and accessible to one or more remote clients via at least one of an application programming interface (API), or an application plug-in.

17. The system of claim 15, wherein the diffusion model is updated with a visual encoder of the 3D compression network.

18. The system of claim 15, wherein the 3D image training data comprises 3D medical image training data, and the diffusion model is updated using input training data comprising one or more text descriptions of the 3D medical image training data, the one or more text descriptions generated using one or more language models.

19. The system of claim 15, wherein the simulated 3D image data comprises at least one of: one or more computed tomography (CT) scans, one or more magnetic resonance imaging (MRI) scans, or one or more positron emission tomography (PET) scans.

20. The system of claim 15, wherein the system is comprised in at least one of:
a system for performing simulation operations;
a system for performing digital twin operations;
a system for performing light transport simulation;
a system for performing collaborative content creation for 3D assets;
a system for performing deep learning operations;
a system for performing remote operations;
a system for performing real-time streaming;
a system for generating or presenting one or more of augmented reality content, virtual reality content, or mixed reality content;
a system implemented using an edge device;
a system for performing conversational AI operations;
a system implementing one or more language models;
a system implementing one or more large language models (LLMs);
a system implementing one or more vision language models (VLMs);
a system implementing one or more multi-modal language models;
a system for generating synthetic data;
a system for generating synthetic data using AI;
a system for performing one or more generative AI operations;
a system incorporating one or more virtual machines (VMs);
a system implemented at least partially in a data center; or
a system implemented at least partially using cloud computing resources.
